(19) United States
(12) Patent Application Publication
(10) Pub. No.: US 2011/0294952 A1
(43) Pub. Date: Dec. 1, 2011

(54) Optimized Drug Conjugates

(75) Inventor: John S. Petersen, Acton, MA (US)

(73) Assignee: SynDevRX, Cambridge, MA (US)

(21) Appl. No.: 13/115,672

(22) Filed: May 25, 2011

Related U.S. Application Data
(60) Provisional application No. 61/482,404, filed on May 4, 2011.
(60) Provisional application No. 61/347,924, filed on May 25, 2010.

(51) Int. Cl. C08F 22/22; C07D 407/08; C07H 21/00; C07K 2/00; C07K 16/00

(52) U.S. Cl. 525/54.1; 530/300; 530/391.1; 536/23.1; 549/332

(57) ABSTRACT

The invention generally relates to optimized drug conjugates.

### RELATED APPLICATIONS

The present application claims the benefit of and priority to each of U.S. provisional application Ser. No. 61/482,404, filed May 4, 2011, and U.S. provisional application Ser. No. 61/347,924, filed May 25, 2010, the content of each of which is incorporated by reference herein in its entirety.

### FIELD OF THE INVENTION

The invention generally relates to optimized drug conjugates.

### BACKGROUND

Many drugs used clinically are limited by a relatively low therapeutic index, owing to toxic side effects. For example, the post-marketing withdrawal of Vioxx and Bextra exemplify the difficulty in assessing and achieving an acceptable therapeutic index. A general belief is that despite significant advances of several independent validation studies, the use of in silico tools must be taken cautiously in the context of their current capability due to the available bioassay data, lack of widespread understanding of model construction and machine learning algorithms (i.e., the black box dilemma), limited chemical space of training data sets, and high potential for multiple mechanisms of drug toxicity that cannot at present be modeled. Valerio (Toxicology and Applied Pharmacology 241 (2009) 356-370). It has recently been stated that “ . . . in-silico ADME-Toxicity predictions vary greatly. Their use in making go/no-go decisions in drug discovery has been limited due to this lack of predictability.” Caldwell (Current Topics in Medicinal Chemistry, 2009, Vol. 9, No. 11).

Targeted drug delivery aims to increase the therapeutic index of a drug by making more drug molecules available at diseased sites while reducing systemic drug exposure. The concept of covalently attaching drugs to water-soluble polymers was first proposed in the mid-1970s (Ringsdorf, J. POLYMER SCI.: Symposium No. 51, 135-153, 1975). In that model, it was envisioned that the pharmacokinetics of the drug attached to the polymeric carrier could be modulated.

Polymer conjugates generally consist of three elements: a polymer, an active moiety, and a linker connecting the active moiety to the polymer. The general strategy for construction of a drug conjugate is to attach an approved drug to a polymer. It is assumed that the optimization performed on the original drug is relevant to performance of the conjugate. It is believed that the linker acts simply as an element of the drug conjugate structure that is used to release the drug. A typical conjugate releases the drug in plasma and the conjugate thus behaves like a slow infusion of the active drug.

While many conjugates have been synthesized and evaluated in animals, few have progressed to clinical trials and those trials have been largely disappointing. The identification of drug conjugates that represent improvements over the parent drug remains an area of active research.

### SUMMARY

It has been discovered that release of an active moiety in a target tissue is a necessary but not sufficient condition to improve efficacy via targeting. For improved efficacy relative to the unconjugated active moiety, the cleavage product must not only be released in the target tissue but it must also exert a substantial portion of its biological effect before transport out of the target tissue, i.e., the equilibration with non-target tissues must be slow relative to biological action in the target tissue. The invention thus provides drug conjugate compositions optimized for adequate influx of the conjugate into a target cell and for reduced or no efflux of the cleaved active moiety from the cell. Reduction in efflux may relate to non-specific diffusion out of a cell or to specific transport out of the cell as mediated, for example, by P-glycoprotein. Reducing efflux increases residence time of the active moiety in the cell (intracellular AUC) and results in improved efficacy. Increased efficacy allows for dose reduction and concomitant reduction in systemic toxicity. Reducing efflux also reduces plasma AUC of the active moiety improving therapeutic index.

The criteria for optimizing the released active moiety from conjugates of the invention are significantly different than the criteria for selecting small molecules for pharmaceutical development. For example, small molecule drugs are generally optimized for enhanced influx into a cell. However, properties of an active moiety that govern influx into a cell also govern efflux from the cell. Optimizing an active moiety to have enhanced influx properties means that the active moiety would also likely have enhanced efflux properties, making it a poor choice as a conjugate that has been designed for intracellular release of the active moiety. Cleaved active moieties of the invention are modified to have reduced efflux from a cell as compared to the unmodified active moiety, making the modified active moiety unsuitable as a small molecule drug due to low influx properties but very suitable for conjugates of the invention. Thus, drug conjugates of the invention, and in particular, active moieties, are optimized for activity in the cell. Accordingly, low doses of the optimized compositions of the invention are used in order to achieve the same or greater therapeutic efficacy as compared to the non-optimized active moiety.

A variety of structural modifications to the cleavage product may be used to control the rate of transport out of the target tissue relative to the rate at which the biological effect is exerted within the tissue. Depending on how the structure is modified, the effect may be to decrease the therapeutic dose, to decrease the toxicity of a therapeutic dose or a combination thereof. The reduction in therapeutic dose and/or reduction in toxicity will result in an improvement in therapeutic index. Released active moiety attributes that may be varied to reduce the efflux include molecular weight, hydrophobicity, polar surface area, and charge. In certain embodiments, these modifications are accomplished by using a linker having a structure such that upon cleavage, a fragment of the linker remains attached to the active moiety which contributes to the desired properties. That fragment may change any of the molecular weight, hydrophobicity, polar surface area, or charge of the active moiety.

Compositions of the invention provide drug conjugates in which the active moiety is selected or modified for reduced efflux from a target tissue compared to other moieties in a family of molecules or an unmodified active moiety. Particularly, drug conjugates of the invention recognize that an active moiety that has been modified for reduced cellular efflux upon intracellular cleavage from the conjugate results in a drug with improved activity and reduced plasma concentration of the active drug in the plasma, i.e., the modified active moiety has certain pharmaceutical properties that are not present in the active moiety, itself. For example, the cleaved active moiety may be inactivated in the target tissue at a higher rate than it is transported out of the target tissue. The modified active moiety may have the effect that the amount of the cleavage product that diffuses away from the target tissue is metabolized to an inactive/low activity species at a greater rate than that of the active moiety alone. The cleaved modified active moiety may be metabolized more rapidly in tissue than its transport rate away from tissue. The modified active moiety may result in a cleavage product that has at least a five-fold greater pharmaceutical activity in the target tissue as compared to the active moiety administered alone (i.e. unconjugated). The modified active moiety may change the toxicity profile of the active moiety, such that the cleavage product has low or no toxicity and/or low or no reactivity in non-target tissue and plasma.

In certain aspects, the invention provides drug conjugate compositions including an active moiety, conjugate moiety, and a cleavable linker, wherein cleavage of the linker occurs substantially in a target tissue and produces a modified active moiety having reduced efflux from target tissue compared to the unmodified active moiety. In other aspects, the invention provides drug conjugate compositions including an active moiety that has low or no capability to enter a cell, a conjugate moiety, and a cleavable linker, wherein cleavage of the linker occurs substantially in a target tissue and the cleaved active moiety is released intra-cellularly.

In certain aspects, the invention provides drug conjugate compositions including an active moiety, a conjugate moiety, and a cleavable linker, wherein cleavage of the linker occurs substantially in a target tissue and produces a modified active moiety that is inactivated in the target tissue at a higher rate than it is transported out of the target tissue. In other aspects, the invention provides drug conjugate compositions including an active moiety, a conjugate moiety, and a cleavable linker, wherein cleavage of the linker in tissue results in a pharmaceutically-active cleavage product and wherein the cleavage product is metabolized more rapidly in tissue than its transport rate away from tissue.

In certain aspects, the invention provides drug conjugate compositions including an active moiety, conjugate moiety, and a cleavable linker, wherein cleavage of the linker produces a modified active moiety having at least about five-fold greater pharmaceutical activity in the tissue as compared to the active moiety alone. In other aspects, the invention provides optimized drug conjugate compositions including an active drug moiety, and a portion of a cleavable linker, in which a large amount of the composition is retained and inactivated in a tissue to which it is targeted, and wherein amounts of the composition that diffuse away from the tissue are metabolized at a greater rate than the active moiety alone. The large amount retained is relative to the amount of small molecule hitting the target in the small molecule case or that it significantly reduce the release of active small molecule relative to concentrations of plasma small molecule generated in the small molecule dosing case. Typically, drug conjugates of the invention allow the use of lower doses of active moiety then would be expected for the active moiety alone due to increased retention of the active moiety in the target.

The linker may be cleaved by any mechanism known in the art. The linker used will depend on the physiological conditions of the target tissue, the properties of the active moiety that are being optimized, and the cleavage mechanism. For example, the linkers may be designed for proteolytic cleavage or intra-cellular proteolytic cleavage.

Any conjugate molecule known in the art may be used with compositions and methods of the invention, and the conjugate used will depend on physiological conditions of the target tissue and the properties of the active moiety. Exemplary conjugates include all forms of polymers, synthetic polymers as well as natural product related polymers including peptides, polysaccharides, polynucleic acids, antibodies and aptamers. In preferable embodiments, the conjugate is a synthetic polymer. Desirable properties of the conjugate include being biocompatible, not accumulating, non-immunogenic, hydrophilic, and biodegradable. In embodiments in which the conjugate is biodegradable, the conjugate degrades at a rate that is slower than the rate of release of the active moiety. In certain embodiments, the conjugate is not biodegradable.

The active moiety may be any compound or molecule that produces a therapeutic effect in a subject. In certain embodiments, the compound or molecule has a molecule weight of about 2000 or less. The compound or molecule chosen will depend on the condition or disease to be treated. In certain embodiments, the active moiety is an anticancer drug. In other embodiments, the active moiety is a molecule that inhibits MetAP2 activity, such as fumagillin, fumagillol, or an analog, derivative, salt or ester thereof. The Journal of Medicinal Chemistry routinely publishes the structure of active moieties that are not suitable for drug development as small molecules because they have poor permeability, low therapeutic index, poor solubility and/or other pharmaceutical limitations but which may be useful for the polymer conjugates of the invention. For example, analogs of Abiraterone, the active moiety released from the prodrug Abiraterone acetate are described by Pinto-Bazurco Mendieta et al. J. Med. Chem 2008, 51 (16), pp 5009-5018 that are useful as CYP17A1 inhibitors. Sunderland et al. J. Med. Chem., 2011, 54 (7), pp 2049-2059 describe a series of 5-benzamidoisoquinolin-1-ones and 5-(ω-carboxyalkyl)isoquinolin-1-ones that are useful as poly(ADP-ribose) polymerase (PARP) inhibitors. Jung et al. J. Med. Chem. 2006, 49, 955-970 describe a series of thiazoloquinazolines that are useful as aurora kinase inhibitors. Njoroge et al. describe the discovery and analog synthesis of the Hepatitis C inhibitor boceprevir in Acc. Chem. Res. 2008, 41 (1), pp 50-59. Lombardo et al. disclose a series of 2-(aminopyridyl)- and 2-(aminopyrimidinyl)thiazole-5-carboxamides which are SRC/Abl kinase inhibitors in J. Med. Chem., 2004, 47 (27), pp 6658-6661.

### BRIEF DESCRIPTION OF THE FIGURES

FIG. 1 is a graph showing percentage weight change as a function of time for C57B1/6 mice, injected initially with B16-F10 tumor cells (1×10⁵), to which one of three polymer conjugates (dosed at 100 mg/kg, q4d) has been administered. Comparative data are included for TNP-470 (dosed at 30 mg/kg, qod) and saline control.

FIG. 2 is a graph showing percentage weight change as a function of time for C57B1/6 mice, injected initially with B16-F10 tumor cells (1×10⁵), to which a polymer conjugate (dosed at 100, 50 and 25 mg/kg, q4d) has been administered. Comparative data are included for TNP-470 (dosed at 30 mg/kg, qod) and saline control.

FIG. 3 is a graph showing the change in tumor size as a function of time for nu/nu mice, injected initially with A549 tumor cells, to which one of three polymer conjugates (dosed at 20 mg/kg, q4d) has been administered. Comparative data are included for TNP-470 (30 mg/kg, qod), a polymer without drug (100 mg/kg, q4d) and saline control.

FIG. 4 is a graph showing the change in body weight change as a function of time for nu/nu mice, injected initially with A549 tumor cells, to which one of three polymer conjugates (dosed at 20 mg/kg, q4d) has been administered. Comparative data are included for TNP-470 (30 mg/kg, qod), a polymer without drug (100 mg/kg, q4d) and saline control.

FIG. 5 is a graph showing the change in tumor size as a function of time for nu/nu mice, injected initially with A549 tumor cells, to which a polymer conjugate (dosed at 6 or 60 mg/kg, q4d) or its active metabolite (dosed at 11 mg/kg q4d) has been administered. Comparative data are included for TNP-470 (30 mg/kg, qod), and a saline control.

FIG. 6 is a graph showing the plasma concentration of the active metabolite, SDX-7539, as a function of time in Sprague-Dawley rats, to which either the polymer conjugate SDX-7320 (single bolus, intravenous injection, 200 mg/kg) or the small molecule SDX-7539 (single bolus, intravenous injection, 30 mg/kg) has been administered.

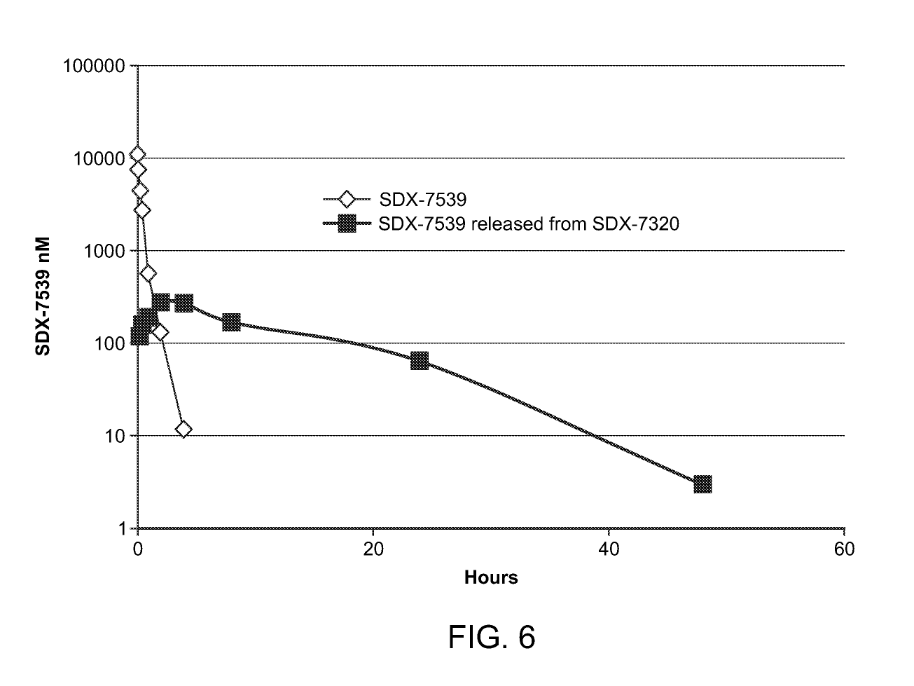

### DETAILED DESCRIPTION

The invention generally relates to optimized drug conjugates. In certain embodiments, the invention provides drug conjugate compositions including an active moiety modified, a conjugate moiety, and a cleavable linker, wherein cleavage of the linker occurs substantially in a target tissue to produce a modified active moiety having reduced efflux from target tissue compared to the unmodified active moiety.

The conjugate moiety used depends on the physicochemical properties of both the conjugate moiety and the active moiety, in addition to biological requirements, e.g., pharmacokinetic and pharmacodynamic properties of the active moiety and knowledge of the disease state. One of skill in the art will be able to select an appropriate conjugate moiety based upon the above considerations. The conjugate moiety may be used to deliver small molecule active moieties or larger molecule active moieties, such as proteins, peptides, or oligonucleotides.

The conjugate moiety improves the delivery of an active moiety to target. The conjugate moiety is chosen to maximize bioavailability of the active moiety, optimize onset, duration, and rate of delivery of the active moiety, and maintain a steady state plasma drug conjugate level within a therapeutic range as long as required for effective treatment. The conjugate moiety may also assist in minimizing adverse side effects of an active moiety. Thus the conjugate moiety prolongs pharmacological activity of an active moiety, stabilizes labile active moieties from chemical and proteolytic degradation, minimizes side effects, increases solubility, and targets the active moiety to specific cells or tissues.

Other properties of the conjugate moiety to be considered are that the conjugate moiety is minimally or non-immunogenic and non-toxic. The molecular weight of the conjugate moiety should be sufficiently large to avoid rapid elimination via kidney ultrafiltration and low enough to prevent undesirable accumulation within the body. In certain embodiments, the conjugate moiety is hydrophilic and is biodegradable. Conjugate moieties that are non-biodegradable are also suitable with compositions and methods of the invention. The conjugate moiety should be able to carry the required amount of active moiety and protect against premature metabolism of the active moiety in transit to the target tissue.

Exemplary conjugates include all forms of polymers, synthetic polymers as well as natural product related polymers including peptides, polysaccharides, polynucleic acids, antibodies and aptamers. In preferable embodiments, the conjugate is a synthetic polymer. Exemplary polymers of the invention have been described in U.S. Pat. No. 4,997,878 to Bock et al, U.S. Pat. No. 5,037,883 to Kopecek et al. U.S. Pat. No. 5,258,453 to Kopecek et al., U.S. Pat. No. 6,464,850 to Zhang et al., U.S. Pat. No. 6,803,438 to Brocchini et al., each of which is incorporated by reference in its entirety. Additional exemplary polymers have been described in Subr et al., J Controlled Release, 18, 123-132 (1992). In some embodiments, the method of synthesis of the polymer may lead to the coupling of two or more polymer chains and may increase the weight average molecular weight of the polymer conjugate. It is further recognized that if this coupling occurs, the linkages will be biodegradable.

In certain embodiments, the conjugate moiety is an antibody. General methodologies for antibody production, including criteria to be considered when choosing an animal for the production of antisera, are described in Harlow et al. (Antibodies, Cold Spring Harbor Laboratory, pp. 93-117, 1988). For example, an animal of suitable size such as goats, dogs, sheep, mice, or camels are immunized by administration of an amount of immunogen effective to produce an immune response. An exemplary protocol is as follows. The animal is injected with 100 milligrams of antigen resuspended in adjuvant, for example Freund's complete adjuvant, dependent on the size of the animal, followed three weeks later with a subcutaneous injection of 100 micrograms to 100 milligrams of immunogen with adjuvant dependent on the size of the animal, for example Freund's incomplete adjuvant. Additional subcutaneous or intraperitoneal injections every two weeks with adjuvant, for example Freund's incomplete adjuvant, are administered until a suitable titer of antibody in the animal's blood is achieved. Exemplary titers include a titer of at least about 1:5000 or a titer of 1:100,000 or more, i.e., the dilution having a detectable activity. The antibodies are purified, for example, by affinity purification on columns containing protein G resin or target-specific affinity resin.

The technique of in vitro immunization of human lymphocytes is used to generate monoclonal antibodies. Techniques for in vitro immunization of human lymphocytes are well known to those skilled in the art. See, e.g., Inai, et al., Histochemistry, 99(5):335 362, May 1993; Mulder, et al., Hum. Immunol., 36(3):186 192, 1993; Harada, et al., J. Oral Pathol. Med., 22(4):145 152, 1993; Stauber, et al., J. Immunol. Methods, 161(2):157 168, 1993; and Venkateswaran, et al., Hybridoma, 11(6) 729 739, 1992. These techniques can be used to produce antigen-reactive monoclonal antibodies, including antigen-specific IgG, and IgM monoclonal antibodies.

In certain embodiments, the conjugate moiety is a aptamer. As used herein, “aptamer” and “nucleic acid ligand” are used interchangeably to refer to a nucleic acid that has a specific binding affinity for a target molecule, such as a protein. Like all nucleic acids, a particular nucleic acid ligand may be described by a linear sequence of nucleotides (A, U, T, C and G), typically 15-40 nucleotides long. Nucleic acid ligands can be engineered to encode for the complementary sequence of a target protein known to associate with the presence or absence of a specific disease.

In solution, the chain of nucleotides form intramolecular interactions that fold the molecule into a complex three-dimensional shape. The shape of the nucleic acid ligand allows it to bind tightly against the surface of its target molecule. In addition to exhibiting remarkable specificity, nucleic acid ligands generally bind their targets with very high affinity, e.g., the majority of anti-protein nucleic acid ligands have equilibrium dissociation constants in the picomolar to low nanomolar range.

Aptamers used in the compositions of the invention depend upon the target tissue. Nucleic acid ligands may be discovered by any method known in the art. In one embodiment, nucleic acid ligands are discovered using an in vitro selection process referred to as SELEX (Systematic Evolution of Ligands by Exponential enrichment). See for example Gold et al. (U.S. Pat. Nos. 5,270,163 and 5,475,096), the contents of each of which are herein incorporated by reference in their entirety. SELEX is an iterative process used to identify a nucleic acid ligand to a chosen molecular target from a large pool of nucleic acids. The process relies on standard molecular biological techniques, using multiple rounds of selection, partitioning, and amplification of nucleic acid ligands to resolve the nucleic acid ligands with the highest affinity for a target molecule. The SELEX method encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. There have been numerous improvements to the basic SELEX method, any of which may be used to discover nucleic acid ligands for use in methods of the invention.

The active moiety may be any compound or molecule that produces a therapeutic effect in a subject. In certain embodiments, the compound or molecule has a molecular weight of 2000 or less. The compound or molecule chosen will depend on the condition or disease to be treated. In certain embodiments, the active moiety is an anticancer drug. In other embodiments, the active moiety is a molecule that inhibits MetAP2 activity, such as fumagillin, fumagillol, or an analog, derivative, salt or ester thereof. The Journal of Medicinal Chemistry routinely publishes the structure of active moieties that are not suitable for drug development as small molecules because they have poor permeability, low therapeutic index, poor solubility and/or other pharmaceutical limitations but which may be useful for the polymer conjugates of the invention. For example, analogs of Abiraterone, the active moiety released from the prodrug Abiraterone acetate are described by Pinto-Bazurco Mendieta et al. J. Med. Chem 2008, 51 (16), pp 5009-5018 that are useful as CYP17A1 inhibitors. Sunderland et al. J. Med. Chem., 2011, 54 (7), pp 2049-2059 describe a series of 5-benzamidoisoquinolin-1-ones and 5-(ω-carboxyalkyl)isoquinolin-1-ones that are useful as poly(ADP-ribose) polymerase (PARP) inhibitors. Jung et al. J. Med. Chem. 2006, 49, 955-970 describe a series of thiazoloquinazolines that are useful as aurora kinase inhibitors. Njoroge et al. describe the discovery and analog synthesis of the Hepatitis C inhibitor boceprevir in Acc. Chem. Res. 2008, 41 (1), pp 50-59. Lombardo et al. disclose a series of 2-(aminopyridyl)- and 2-(aminopyrimidinyl)thiazole-5-carboxamides which are SRC/Abl kinase inhibitors in J. Med. Chem., 2004, 47 (27), pp 6658-6661.

In certain embodiments, the active moiety is an anticancer drug. In other embodiments, the active moiety is a molecule that inhibits MetAP2 activity, such as fumagillin, fumagillol, or an analog, derivative, salt or ester thereof. Further exemplary MetAP2 inhibitors have been described in U.S. Pat. No. 6,242,494 to Craig et al, U.S. Pat. No. 6,063,812 to Hong et al., U.S. Pat. No. 6,887,863 to Craig et al., U.S. Pat. No. 7,030,262 to BaMaung et al., U.S. Pat. No. 7,491,718 to Comess et al., each of which is incorporated by reference in its entirety. Additional exemplary MetAP2 inhibitors have been described in Wang et al. “Correlation of tumor growth suppression and methionine aminopeptidase-2 activity blockade using an orally active inhibitor,” PNAS 105(6) 1838-1843 (2008); Lee at al. “Design, Synthesis, and Antiangiogenic Effects of a Series of Potent Novel Fumagillin Analogues,” Chem. Pharm. Bull. 55(7) 1024-1029 (2007); Jeong et al. “Total synthesis and antiangiogenic activity of cyclopentane analogues of fumagillol,” Bioorganic and Medicinal Chemistry Letters 15, 3580-3583 (2005); Arico-Muendel et al. “Carbamate Analogues of Fumagillin as Potent, Targeted Inhibitors of Methionine Aminopeptidase-2,” J. Med. Chem. 52, 8047-8056 (2009); and International Publication No. WO 2010/003475 to Heinrich et al.

The MetAP2 inhibitors described herein possess broad therapeutic benefits including metabolic, anti-proliferative and anti-angiogenic activity. As angiogenesis inhibitors, such compounds are useful in the treatment of both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder, and urothelium), female genital tract (including cervix, uterus, and ovaries as well as choriocarcinoma and gestational trophoblastic disease), male genital tract (including prostate, seminal vesicles, testes, and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). Such compounds may also be useful in treating solid tumors arising from hematopoietic malignancies such as leukemias (i.e., chloromas, plasmacytomas and the plaques and tumors of mycosis fungosides and cutaneous T-cell lymphoma/leukemia) as well as in the treatment of lymphomas (both Hodgkin's and non-Hodgkin's lymphomas). In addition, these compounds may be useful in the prevention of metastases from the tumors described above either when used alone or in combination with radiotherapy and/or other chemotherapeutic agents. The compounds of the invention can also be useful in the treatment of the aforementioned conditions by mechanisms other than the inhibition of angiogenesis.

Further uses include the treatment and prophylaxis of diseases such as blood vessel diseases such as hemangiomas, and capillary proliferation within atherosclerotic plaques; Osler-Webber Syndrome; myocardial angiogenesis; plaque neovascularization; telangiectasia; hemophiliac joints; angiofibroma; and wound granulation. Other uses include the treatment of diseases characterized by excessive or abnormal proliferation of endothelial cells, including not limited to intestinal adhesions, Crohn's disease, atherosclerosis, scleroderma, and hypertrophic scars, i.e., keloids. Another use is as a birth control agent, by inhibiting ovulation and establishment of the placenta. The compounds of the invention are also useful in the treatment of diseases that have angiogenesis as a pathologic consequence such as cat scratch disease (Rochele minutesalia quintosa) and ulcers (Helicobacter pylori). The compounds of the invention are also useful to reduce bleeding by administration prior to surgery, especially for the treatment of resectable tumors.

In compositions of the invention, the conjugate moiety is joined to the active moiety via a linker. Any linker structure known in the art may be used to join the modified active moiety to the conjugate moiety. The linker used will depend on the physiological conditions of the target tissue, the properties of the active moiety that are being optimized, and the cleavage mechanism. D'Souza et al. review various types of linkers including linkers that operate via proteolytic cleavage “Release from Polymeric Prodrugs: Linkages and Their Degradation” J. Pharm. Sci., 93, 1962-1979 (2004). Blencoe et al. describe a variety of self-immolative linkers, “Self-immolative linkers in polymeric delivery systems” Polym. Chem. 2, 773-790 (2011). Ducry et al. review linkers in Bioconj. Chem. 21, 5-13 (2010) “Antibody-Drug Conjugates: Linking Cytotoxic Payloads to Monoclonal Antibodies”. Other linker chemistries suitable with compositions of the invention are shown in Shiose et al. Biol. Pharm. Bull. 30(12) 2365-2370 (2007); Shiose et al. Bioconjugate Chem. 20(1) 60-70 (2009); Senter, U.S. Pat. No. 7,553,816; De Groot, U.S. Pat. No. 7,223,8371; King, U.S. Pat. No. 6,759,509; Susaki, U.S. Pat. No. 6,835,807; and Susaki U.S. Pat. No. 6,436,912.

In certain embodiments, the linker is a peptide linker. Exemplary peptide linkers are described in U.S. Pat. No. 6,835,807 to Susaki et al., U.S. Pat. No. 6,291,671 to Inoue et al., U.S. Pat. No. 6,811,996 to Inoue et al., U.S. Pat. No. 7,041,818 to Susaki et al., U.S. Pat. No. 7,091,186 to Senter et al., U.S. Pat. No. 7,553,816 to Senter et al. each of which is incorporated by reference in its entirety. Additional exemplary peptides and their cleavage have been described in Shiose et al. Biol. Pharm. Bull. 30(12) 2365-2370 (2007) and Shiose et al. Bioconjugate Chem. 20(1) 60-70 (2009). Peptide linkers suitable for cleavage by matrix metalloproteins (MMPs) are described in Chau et al. “Antitumor efficacy of a novel polymer-peptide-drug conjugate in human tumor xenograft models” Int. J. Cancer 118, 1519-1526 (2006) and Chau et al. U.S. patent publication number 2004/0116348.

The linker may be cleaved by any mechanism known in the art. For example, the linkers may be designed for proteolytic cleavage or intracellular proteolytic cleavage. In certain embodiments, the linker is designed such that there is no cleavage of the linker in plasma or there is a very low rate of cleavage in the plasma. Exemplary linker structures are described in further detail below.

In certain embodiments, the linker has a structure such that it is to be preferentially cleaved in disease tissue. Since most hydrolases exist in both normal and diseased tissue, the linker should be cleaved by a hydrolase that is more active in disease tissue and/or more prevalent in disease tissue. For example, tumors have generally upregulated metabolic rates and in particular over express proteases including the cathepsins. The upregulation and role of proteases in cancer is described by Mason et al. Trends in Cell Biology 21, 228-237 (2011).

In any hydrolysis process, one of the cleaved entities will add a hydroxyl group and the other will add hydrogen. Conjugates of the invention may have either orientation of the cleavable functionality. For example, a conjugate of the invention containing the cleavable group Y—X which is part of a linker L1-L2 may be cleaved as in the general formula I where the cleavage product (active drug) bears a hydrogen atom and formula II where the specific case of amide cleavage is exemplified.

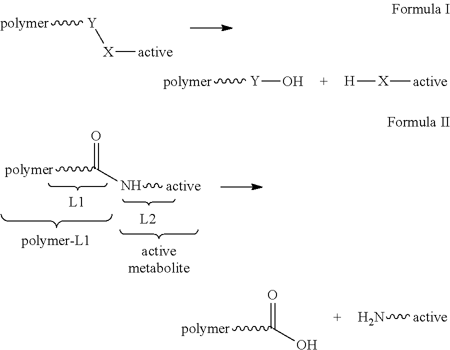

Alternatively the linker may be oriented so that the cleavage product bears a hydroxyl group, as shown in formulas III and IV below.

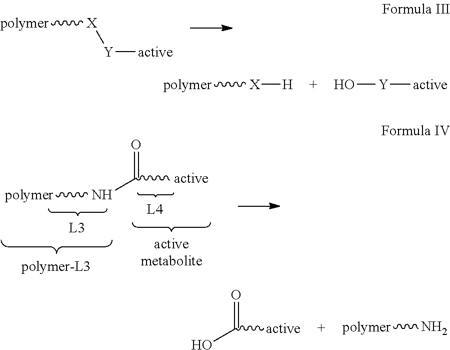

Linkers that are stable in plasma are preferred as plasma release of the active small molecule will not show a therapeutic advantage relative to slow direct administration of the small molecule.

The invention recognizes that release of an active moiety in a target tissue is a necessary but not sufficient condition to improve efficacy via targeting. For improved efficacy relative to the parent active moiety, the cleavage product must not only be released in the target tissue but it must also exert a substantial portion of its biological effect before transport out of the target tissue i.e., the equilibration with non-target tissues must be slow relative to biological action in the target tissue.

A variety of structural modifications to the cleavage product may be used to control the rate of transport out of the target tissue relative to the rate at which the biological effect is exerted within the tissue. Depending on how the structure is modified, the effect may be to decrease the therapeutic dose, to decrease the toxicity of a therapeutic dose or a combination thereof. The reduction in therapeutic dose and/or reduction in toxicity will result in an improvement in therapeutic index. Released active moiety attributes that may be varied to reduce the efflux include molecular weight, hydrophobicity, polar surface area, and charge.

Ertl et al. show that increased polar surface area results in reduced absorption “Fast Calculation of Molecular Polar Surface Area as a Sum of Fragment-Based Contributions and Its Application to the Prediction of Drug Transport Properties” J. Med. Chem., 43, 3714-3717 (2000). Thus an increase in polar surface area may be used to reduce efflux. vanDe Waterbeemd et al. show that selection of active moieties with lower molecular weight tends to decrease permeability “Estimation of Caco-2 Cell Permeability using Calculated Molecular Descriptors” Quant. SAR 15, 480-490, (1996). Introduction of a cationic functional group may also be used to reduce permeation Palm et al. J. Pharm. Exp. Ther. 291, 435-443 (1999). Decreasing hydrophobicity is also correlated with reduced permeability Di et al. Curr. Pharm. Design 15, 2184-2194 (2009).

Compositions of the invention provide drug conjugates in which the cleaved active moiety is modified for reduced efflux from a target tissue compared to an unmodified active moiety. Alternatively, the cleaved active moiety is selected from a family of active moieties that have comparable target affinities but the selected member has reduced efflux compared to other members of the family. Particularly, drug conjugates of the invention recognize that an active moiety that has been modified for reduced cellular efflux upon intracellular cleavage from the conjugate results in a drug with improved activity and reduced plasma concentration of the active drug in the plasma, i.e., the modified active moiety has certain pharmaceutical properties that are not present in solely the active moiety. For example, the modified active moiety may be inactivated in the target tissue at a higher rate than it is transported out of the target tissue. The modified active moiety may have the effect that the amount of the cleavage product that diffuses away from the target tissue is metabolized at a greater rate than that of the active moiety alone. The modified active moiety may be metabolized more rapidly in target tissue than its transport rate away from target tissue. The modified active moiety may result in a cleavage product that has at least a five-fold greater pharmaceutical activity in the target tissue as compared to the active moiety alone. The modified active moiety may change the toxicity profile of the active moiety, such that the cleavage product has low or no toxicity and/or low or no reactivity in non-target tissue and plasma.

In certain embodiments, the class of active moieties that are modified are moieties that irreversibly bind to their targets, i.e., after release from the conjugate the active moiety covalently binds to the biochemical target. Once bound, the active moiety cannot diffuse or be transported out of the cell. For targeting to occur in the case of irreversible binding, the rate of small molecule binding to target, kᵢᵣᵣₑᵥ, should be significant relative to the rate of small molecule efflux, kₛₘ₋₁. If the rate of efflux is high relative to small molecule binding, small molecule equilibrium will be established between the plasma and the intracellular compartment and there will be no advantage to intracellular delivery relative to extracellular delivery. Such a relationship is described in formula V below, where: [PC]=concentration of polymer conjugate; [SM]=concentration of released small molecule; plasma=plasma concentration; icell=intracellular concentration; icell-target=small molecule irreversibly bound to intracellular target; and inactive=inactive metabolite of small molecule.

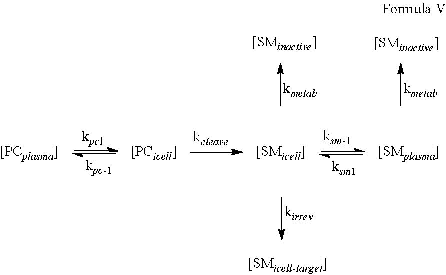

In other embodiments, the class of active moieties that are modified are moieties that reversibly bind to their targets. For targeting to occur in the case of reversible binding, the equilibrium constant for small molecule binding to target K=kᵣₑᵥ₁/kᵣₑᵥ₋₁ should be large and the “on-rate”, kᵣₑᵥ₁, should be large relative to the rate of small molecule efflux, kₛₘ₋₁. If the rate of efflux is high relative to small molecule binding, small molecule equilibrium will be established between the plasma and the intracellular compartment and there will be no advantage to intracellular delivery relative to extracellular delivery. Such a relationship is described in formula VI below, where: [PC]=concentration of polymer conjugate; [SM]=concentration of released small molecule; plasma=plasma concentration; icell=intracellular concentration; icell-target=small molecule reversibly bound to intracellular target; and inactive=inactive metabolite of small molecule.

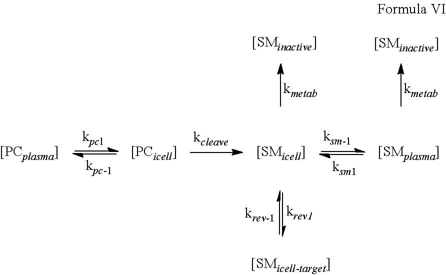

In other embodiments, the class of active moieties that are modified are moieties that have very high equilibrium constants and high “on-rates” relative to efflux. In other embodiments, the class of active moieties that are modified are moieties that undergo intracellular metabolism at a high rate relative to efflux.

In certain embodiments, modifications to the active moiety are accomplished by using a linker having a structure such that upon cleavage, a fragment of the linker remains attached to the active moiety. That fragment may change any of the molecular weight, hydrophobicity, polar surface area, or charge of the active moiety, thereby producing a modified active moiety having reduced efflux from a target cell compared to the unmodified active moiety. For example, coupling MetAP2 inhibitory active moieties via the linkers described herein provide conjugates in which upon cleavage of the linker, produce an active moiety having a fragment of the linker attached thereto (modified active moiety). The modified active moieties described herein have reduced efflux from a cell compared to the unmodified active moieties, resulting in modified active moieties with superior efficacy to the parent small molecules and superior pharmacokinetic profiles. One aspect of the present invention provides conjugates with linkers having the structure:

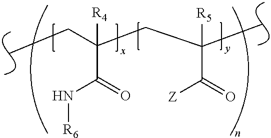

wherein, independently for each occurrence, R₄ is H or C₁-C₆ alkyl; R₅ is H or C₁-C₆ alkyl; R₆ is C₂-C₆ hydroxyalkyl; Z is —NH-AA₁-AA₂-AA₃-AA₄-AA₅-AA₆-C(O)-L or —NH-AA₁-AA₂-AA₃-AA₄-AA₅-AA₆-C(O)-Q-X—Y—C(O)—W; AA₁ is glycine, alanine, or H₂N(CH₂)mCO₂H, wherein is 2, 3, 4 or 5; AA₂ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine; AA₃ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine; AA₄ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine; AA₅ is a bond, or glycine, valine, tyrosine, tryptophan, phenylalanine, methionine, leucine, isoleucine, or asparagine; AA₆ is a bond, or alanine, asparagine, citrulline, glutamine, glycine, leucine, methionine, phenylalanine, serine, threonine, tryptophan, tyrosine, valine, or H₂N(CH₂)mCO₂H, wherein m is 2, 3, 4 or 5; L is —OH, —O-succinimide, —O-sulfosuccinimide, alkoxy, aryloxy, acyloxy, aroyloxy, alkoxycarbonyloxy, aryloxycarbonyloxy, —NH₂, —NH(C₂-C₆ hydroxyalkyl), halide or perfluoroalkyloxy; Q is NR, O, or S; X is M-(C(R)₂)ₚ-M-J-M-(C(R)₂)ₚ-M-V; M is a bond, or C(O); J is a bond, or ((CH₂)_{q}Q)ᵣ, C₅-C₈ cycloalkyl, aryl, heteroaryl, NR, O, or S; Y is NR, O, or S; R is H or alkyl; V is a bond or

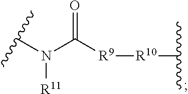

R⁹ is alkyl, aryl, aralkyl, or a bond; or R⁹ taken together with Y forms a heterocyclic ring; R¹⁰ is amido or a bond; R¹¹ is H or alkyl; W is a MetAP2 inhibitor moiety or alkyl; x is in the range of 1 to about 450; y is in the range of 1 to about 30; n is in the range of 1 to about 50; p is 0 to 20; q is 2 or 3; and r is 1, 2, 3, 4, 5, or 6.

In certain embodiments, R₄ is C₁-C₆ alkyl. In certain embodiments, R₄ is methyl. In certain embodiments, R₅ is C₁-C₆ alkyl. In certain embodiments, R₅ is methyl. In certain embodiments, R₆ is 2-hydroxyethyl, 2-hydroxypropyl or 3-hydroxypropyl. In certain embodiments, R₆ is 2-hydroxypropyl.

In certain embodiments, the compound has a molecular weight of less than about 60 kDa. In other embodiments, the molecular weight is less than about 45 kDa. In other embodiments, the molecular weight is less than about 35 kDa.

In certain embodiments, the ratio of x to y is in the range of about 30:1 to about 3:1. In other embodiments, the ratio of x to y is in the range of about 19:2 to about 7:2. In certain embodiments, the ratio of x to y is in the range of about 9:1 to about 4:1. In certain embodiments, the ratio of x to y is about 11:1. In certain embodiments, the ratio of x to y is about 9:1. In certain embodiments, the ratio of x to y is about 4:1.

In certain embodiments, Z is —NH-AA₁-AA₂-AA₃-AA₄-AA₅-AA₆-C(O)-L. In certain embodiments, L is methoxy, ethoxy, pentafluorophenyloxy, phenyloxy, acetoxy, fluoride, chloride, methoxycarbonyloxy; ethoxycarbonyloxy, phenyloxycarbonyloxy, 4-nitrophenyloxy, trifluoromethoxy, pentafluoroethoxy, or trifluoroethoxy. In certain embodiments, L is 4-nitrophenyloxy.

In certain embodiments, Z is —NH-AA₁-AA₂-AA₃-AA₄-AA₅-AA₆-C(O)-Q-X—Y—C(O)—W. In certain embodiments, AA₁ is glycine. In certain embodiments, AA₂ is glycine. In certain embodiments, AA₃ is glycine. In certain embodiments, AA₄ is glycine or phenylalanine. In certain embodiments, AA₅ is leucine, phenylalanine, valine or tyrosine. In certain embodiments, AA₆ is asparagine, citrulline, glutamine, glycine, leucine, methionine, threonine or tyrosine. In certain embodiments, AA₅-AA₆ is Leu-Cit, Leu-Gln, Leu-Gly, Leu-Leu, Leu-Met, Leu-Thr, Phe-Cit, Phe-Gln, Phe-Leu, Phe-Met, Phe-Thr, Val-Asn, Val-Cit, Val-Gln, Val-Leu, Val-Met, Val-Thr, Tyr-Cit, Tyr-Leu, or Tyr-Met. In certain embodiments, AA₁, AA₃ and AA₅ are glycine, valine, tyrosine, tryptophan, phenylalanine, methionine, leucine, isoleucine, or asparagine. In certain embodiments, AA₂, AA₄ and AA₆ are glycine, asparagine, citrulline, glutamine, glycine, leucine, methionine, phenylalanine, threonine or tyrosine. In certain embodiments, AA₂ is a bond; and AA₃ is a bond. In certain embodiments, AA₁ is glycine; AA₄ is phenylalanine; AA₅ is leucine; and AA₆ is glycine.

In certain embodiments, W is

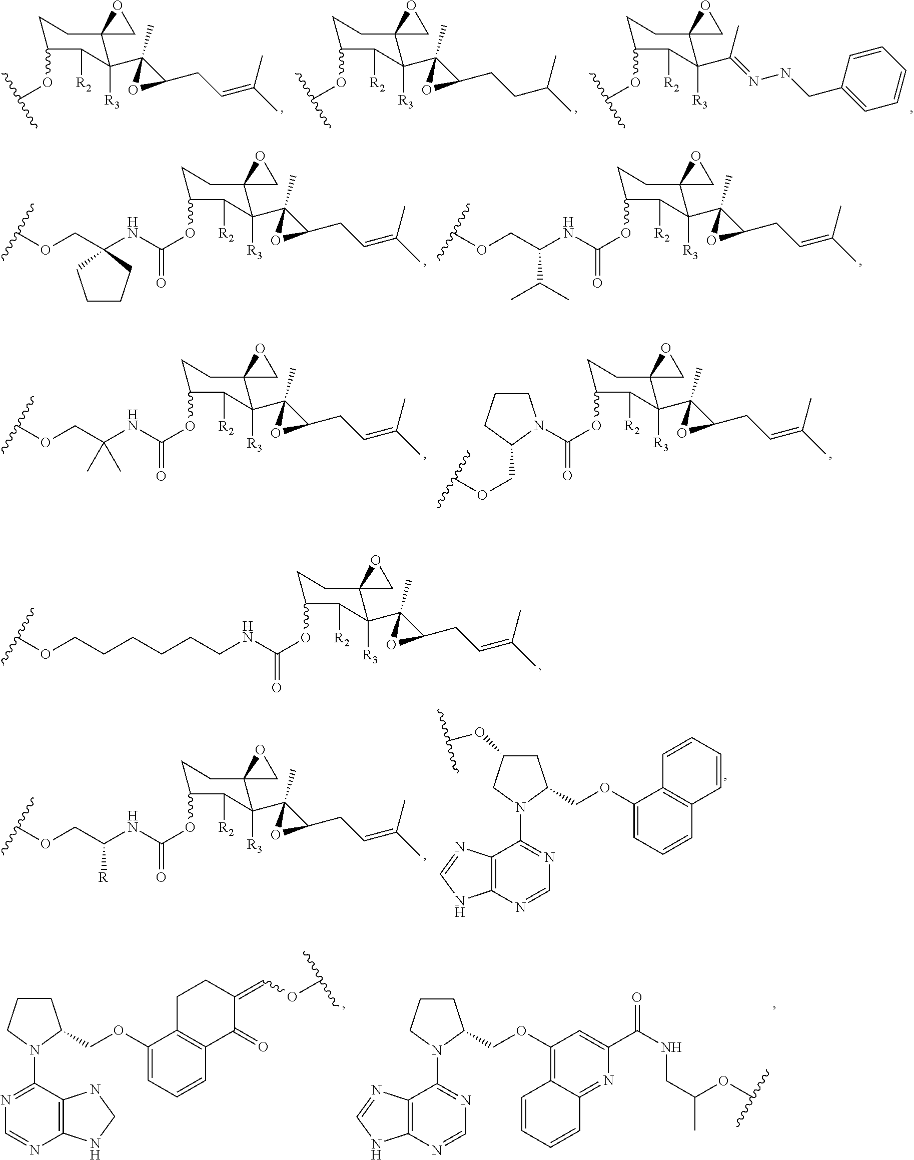

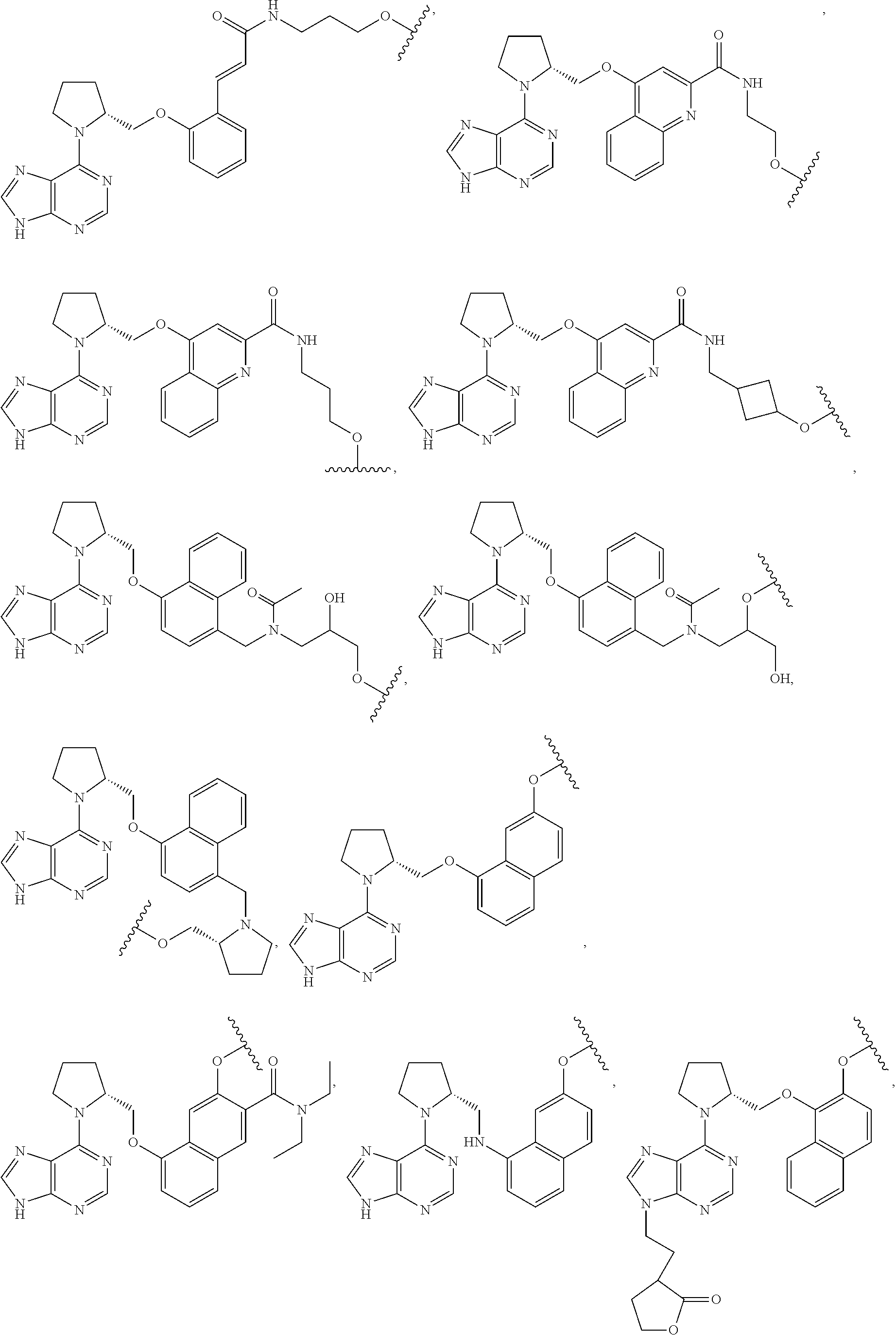

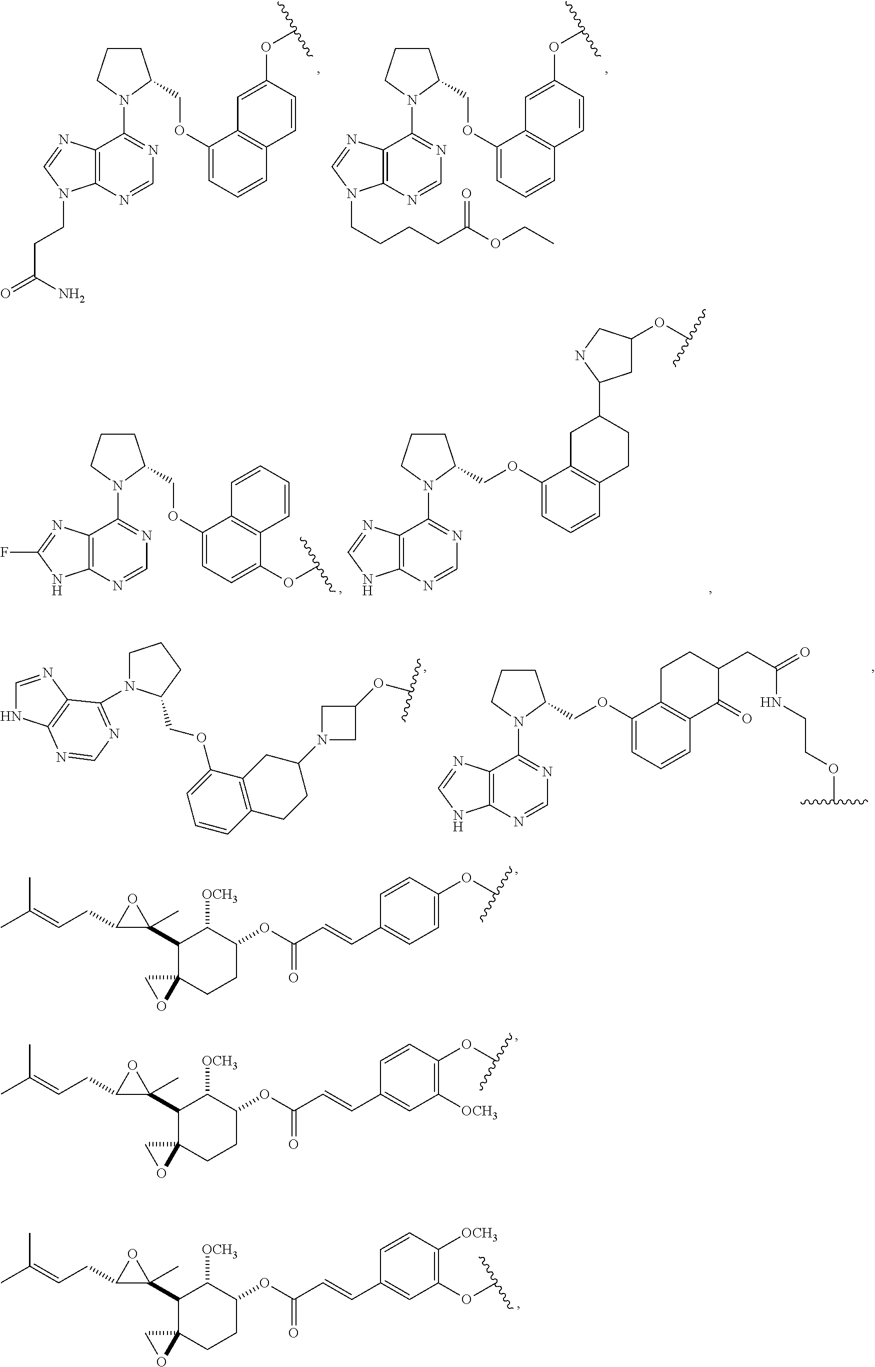

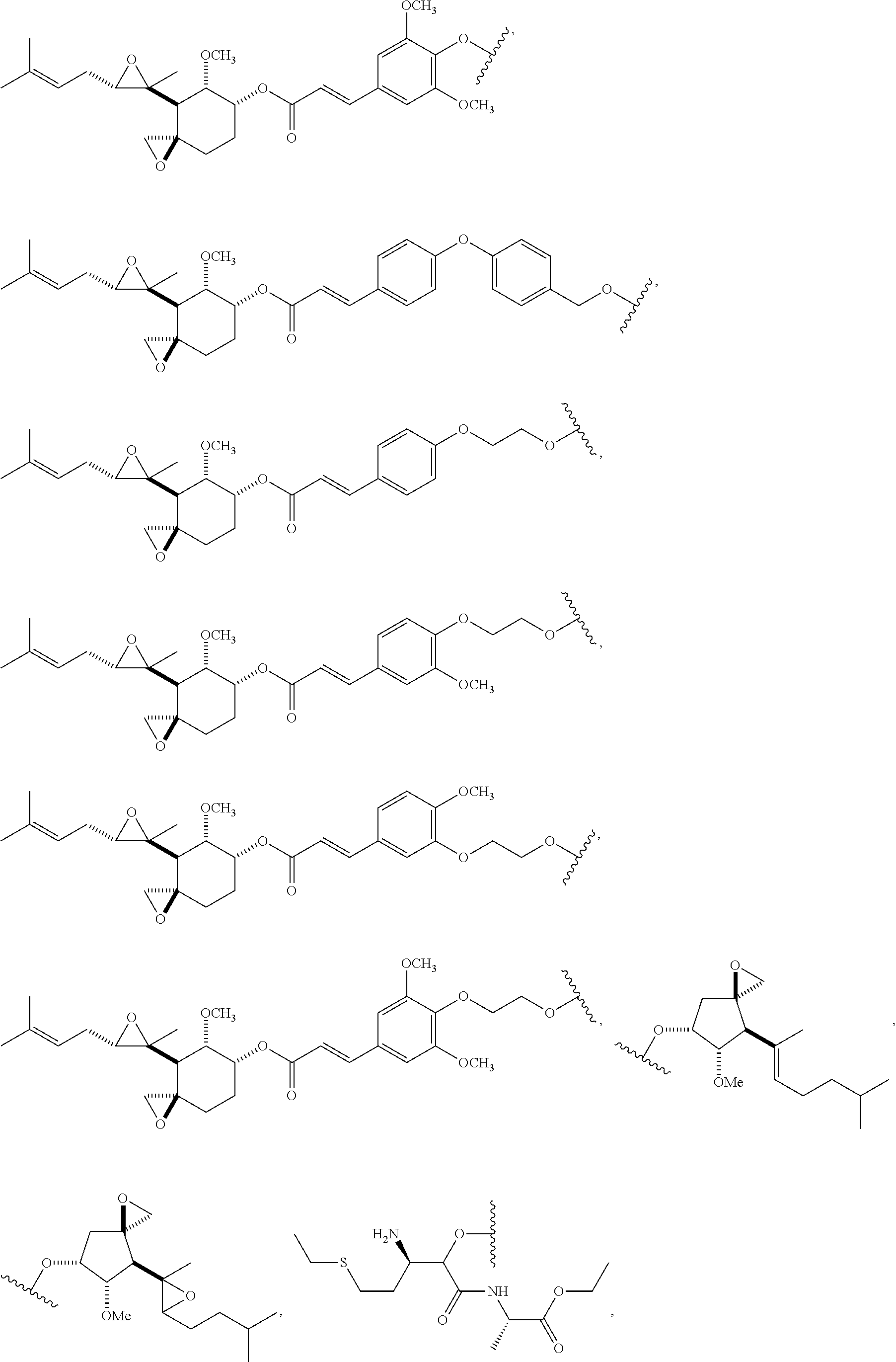

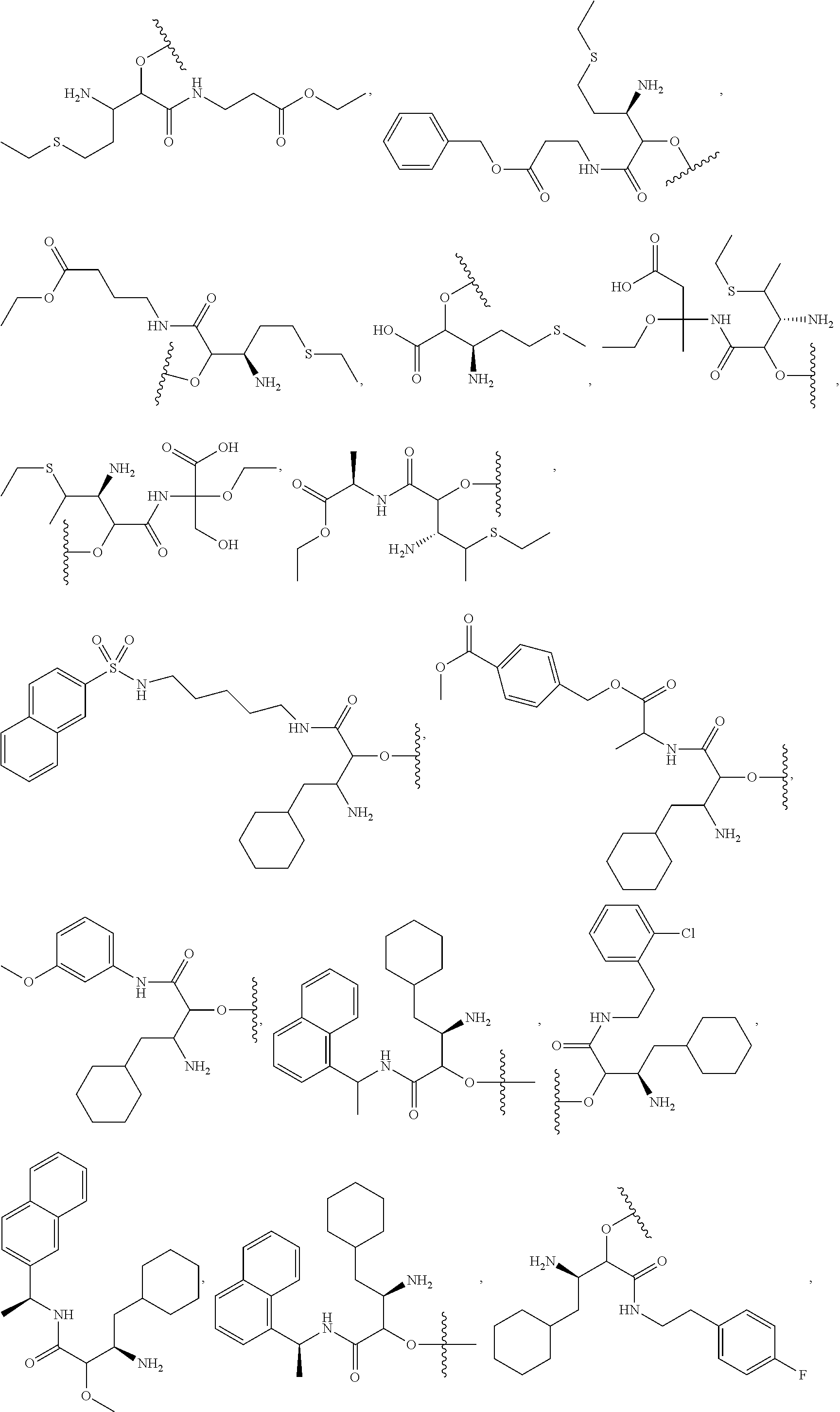

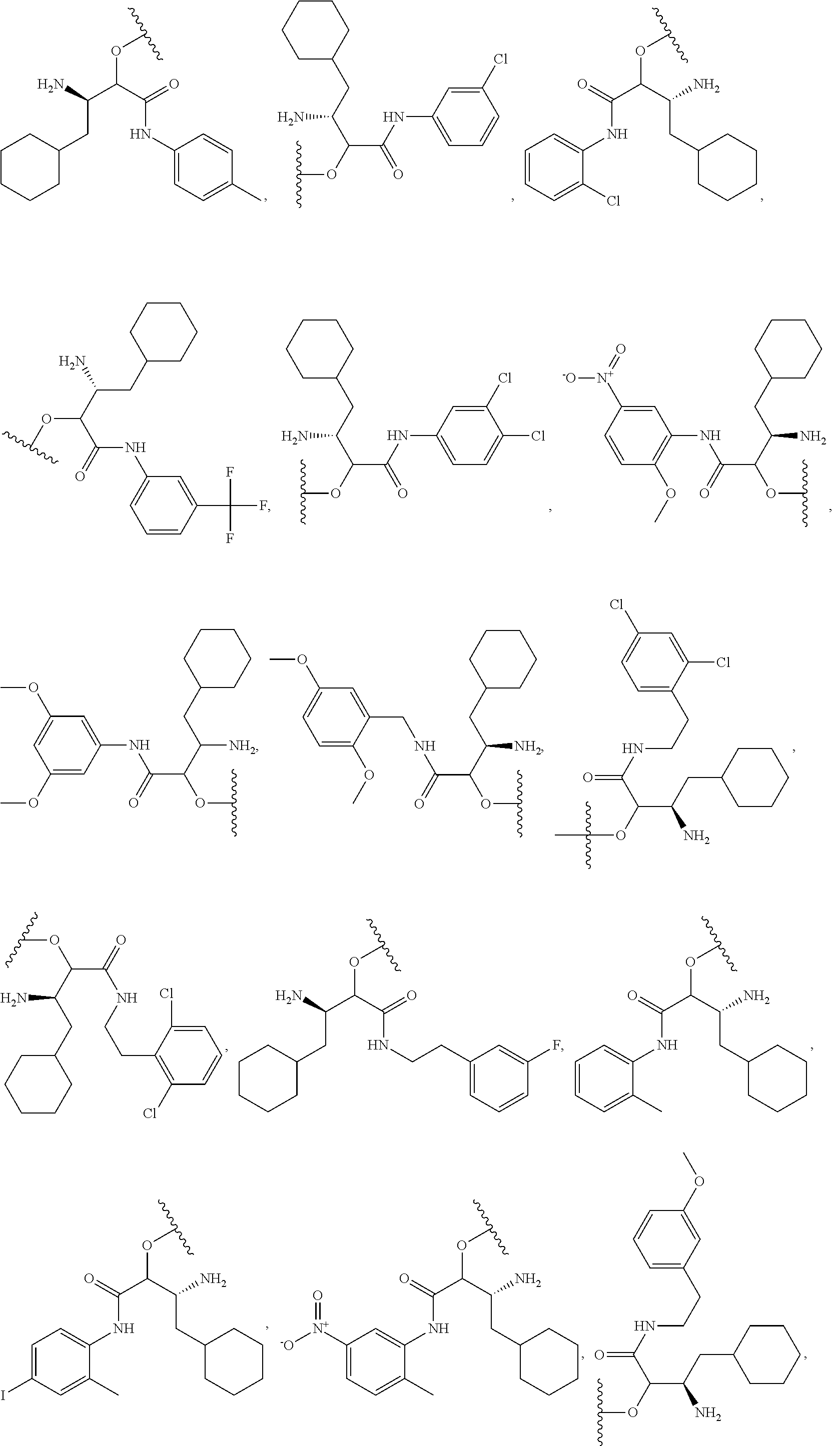

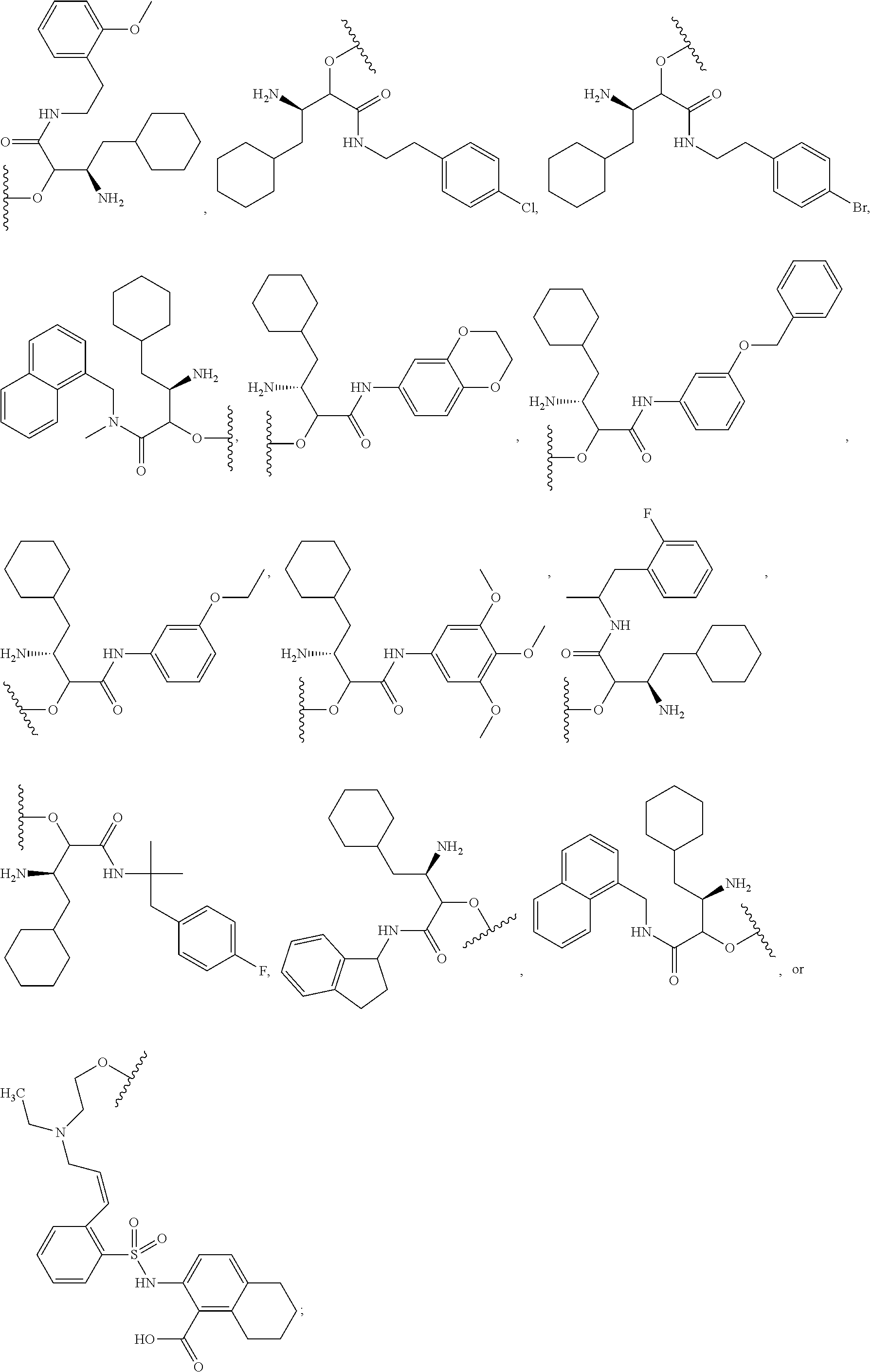

wherein R₂ is —OH or methoxy; and R₃ is H, —OH or methoxy.

In certain embodiments, W is

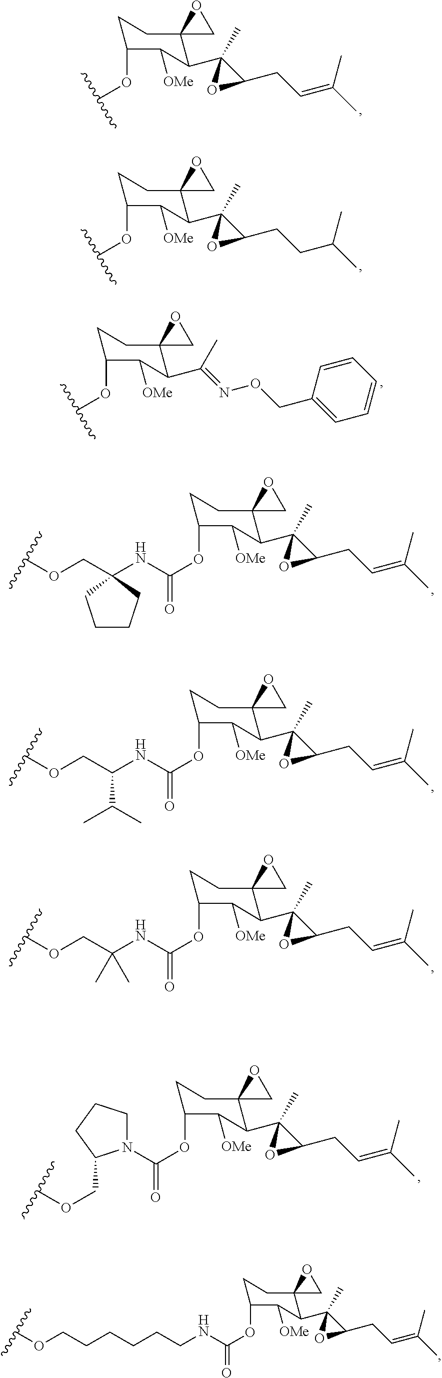

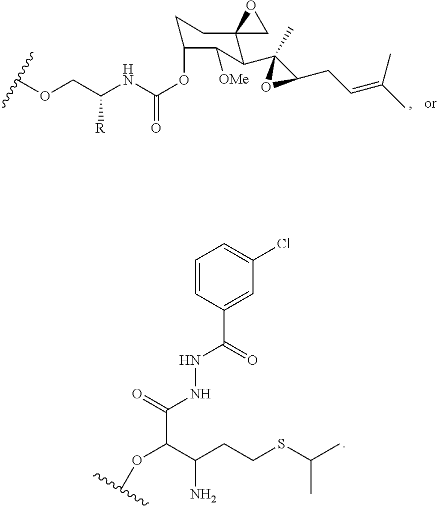

In certain embodiments, W is

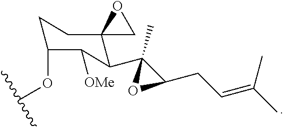

In certain embodiments, Q is NR. In other embodiments, Q is S.

In certain embodiments, J is NR. In other embodiments, J is ((CH₂)_{q}Q)ᵣ. In other embodiments, J is C₅-C₈ cycloalkyl. In certain embodiments, J is aryl.

In certain embodiments, Y is NR. In other embodiments, Y is S.

In certain embodiments, -Q-X—Y— is

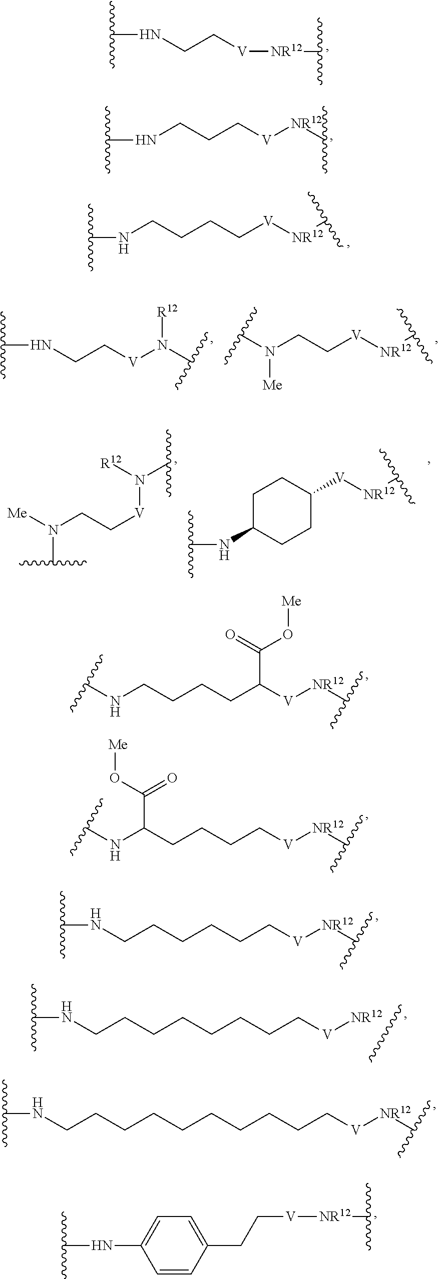

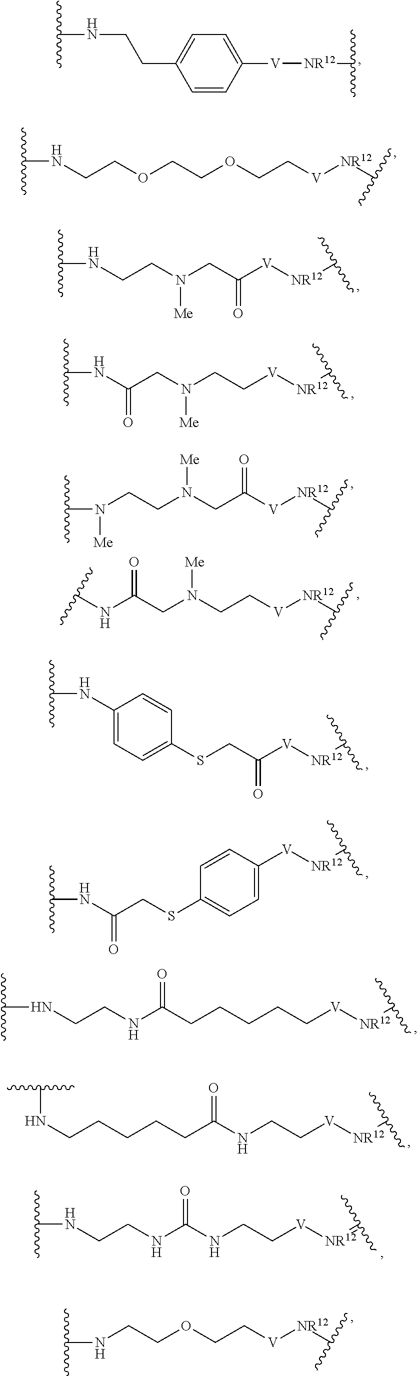

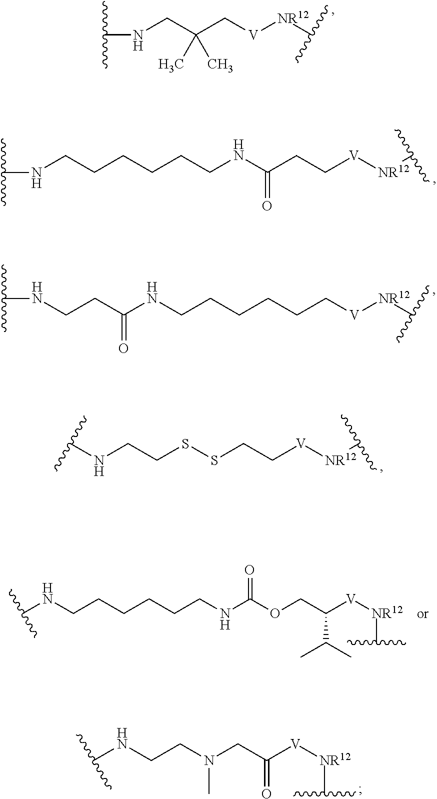

### V is:

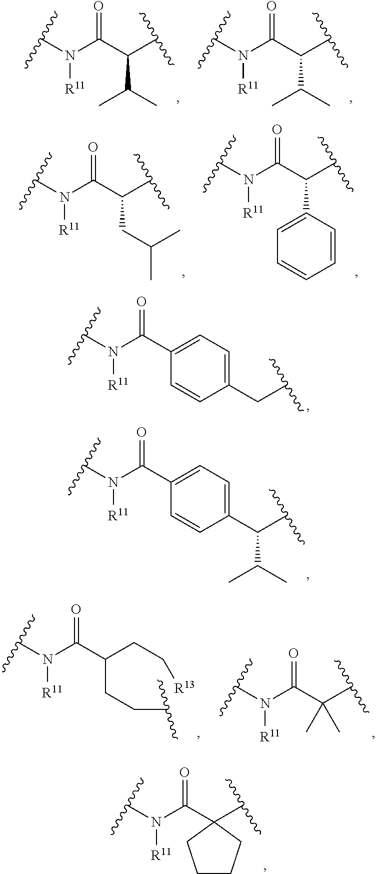

or a bond; R¹² is H or Me; or R¹² taken together with R¹⁴ forms a piperidine ring; R¹¹ is H or Me; and R¹³ taken together with R¹² forms a piperidine ring.

In certain embodiments, -Q-X—Y— is

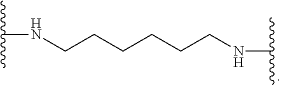

In certain embodiments, -Q-X—Y— is

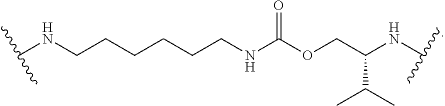

In certain embodiments, -Q-X—Y— is

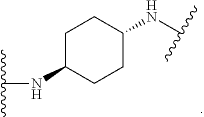

In certain embodiments, -Q-X—Y— is

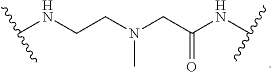

In certain embodiments, R₄ and R₅ are methyl; R₆ is 2-hydroxypropyl; Z is —NH-AA₁-AA₂-AA₃-AA₄-AA₅-AA₆-C(O)-Q-X—Y—C(O)—W; AA₁ is glycine; AA₂ is a bond; AA₃ is a bond; AA₄ is phenylalanine; AA_{S} is leucine; AA₆ is glycine; -Q-X—Y— is

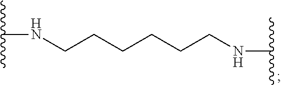

### and W is

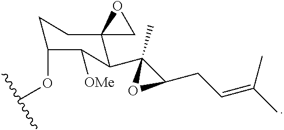

In certain embodiments, R₄ and R₅ are methyl; R₆ is 2-hydroxypropyl; Z is —NH-AA₁-AA₂-AA₃-AA₄-AA₅-AA₆-C(O)-Q-X—Y—C(O)—W; AA₁ is glycine; AA₂ is a bond; AA₃ is a bond; AA₄ is phenylalanine; AA₅ is leucine; AA₆ is glycine; -Q-X—Y— is

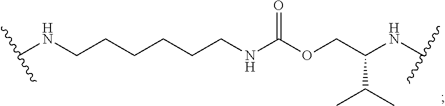

### and W is

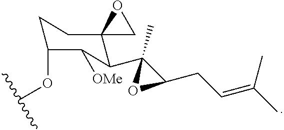

In certain embodiments, R₄ and R₅ are methyl; R₆ is 2-hydroxypropyl; Z is —NH-AA₁-AA₂-AA₃-AA₄-AA₅-AA₆-C(O)-Q-X—Y—C(O)—W; AA₁ is glycine; AA₂ is a bond; AA₃ is a bond; AA₄ is phenylalanine; AA₅ is leucine; AA₆ is glycine; -Q-X—Y— is

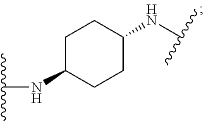

### and W is

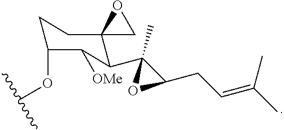

In certain embodiments, -Q-X—Y— is a self-immolating linker that releases the MetAP2 inhibitor in the form of a carbamate derivative, as shown in the scheme below:

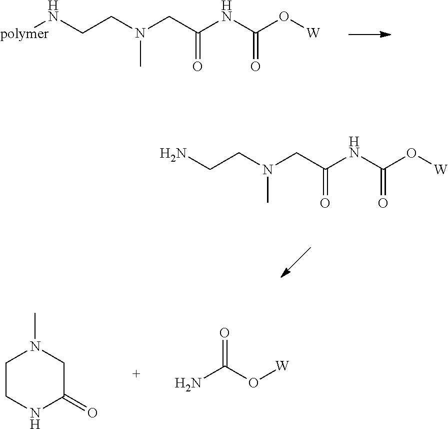

Another aspect of the present invention provides conjugates with linkers having the structure: Z-Q-X—Y—C(O)—W; wherein, independently for each occurrence, Z is H₂N-AA₆-C(O)— or H; AA₆ is alanine, asparagine, citrulline, glutamine, glycine, leucine, methionine, phenylalanine, serine, threonine, tryptophan, tyrosine, valine or H₂N(CH₂)mCO₂H, wherein m is 2, 3, 4 or 5; Q is NR, O, or S; X is M-(C(R)₂)ₚ-M-J-M-(C(R)₂)ₚ-M-V; M is a bond, or C(O); J is a bond, or ((CH₂)_{q}Q)ᵣ, C₅-C₈ cycloalkyl, aryl, heteroaryl, NR, O, or S; Y is NR, O, or S; R is H or alkyl; V is a bond or

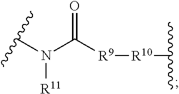

R⁹ is alkyl, aryl, aralkyl, or a bond; or R⁹ taken together with Y forms a heterocyclic ring; R¹⁰ is amido or a bond; R¹¹ is H or alkyl; W is a MetAP2 inhibitor moiety; p is 0 to 20; q is 2 or 3; and r is 1, 2, 3, 4, 5, or 6.

In certain embodiments, Z is H. In other embodiments, Z is H₂N-AA₆-C(O)—. In certain embodiments, AA₆ is glycine. In certain embodiments, Q is NR. In certain embodiments, M is a bond. In certain embodiments, J is a bond. In certain embodiments, Y is NR.

In certain embodiments, W is:

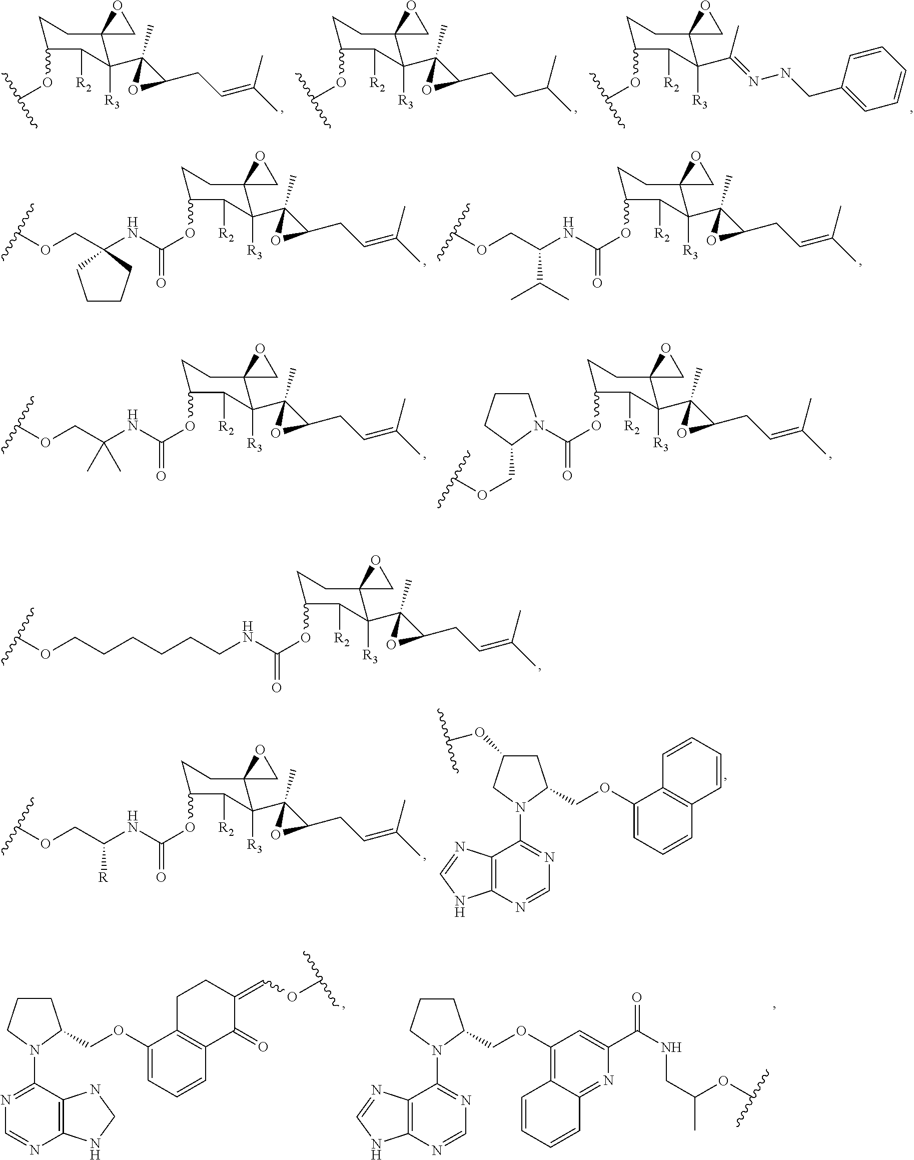

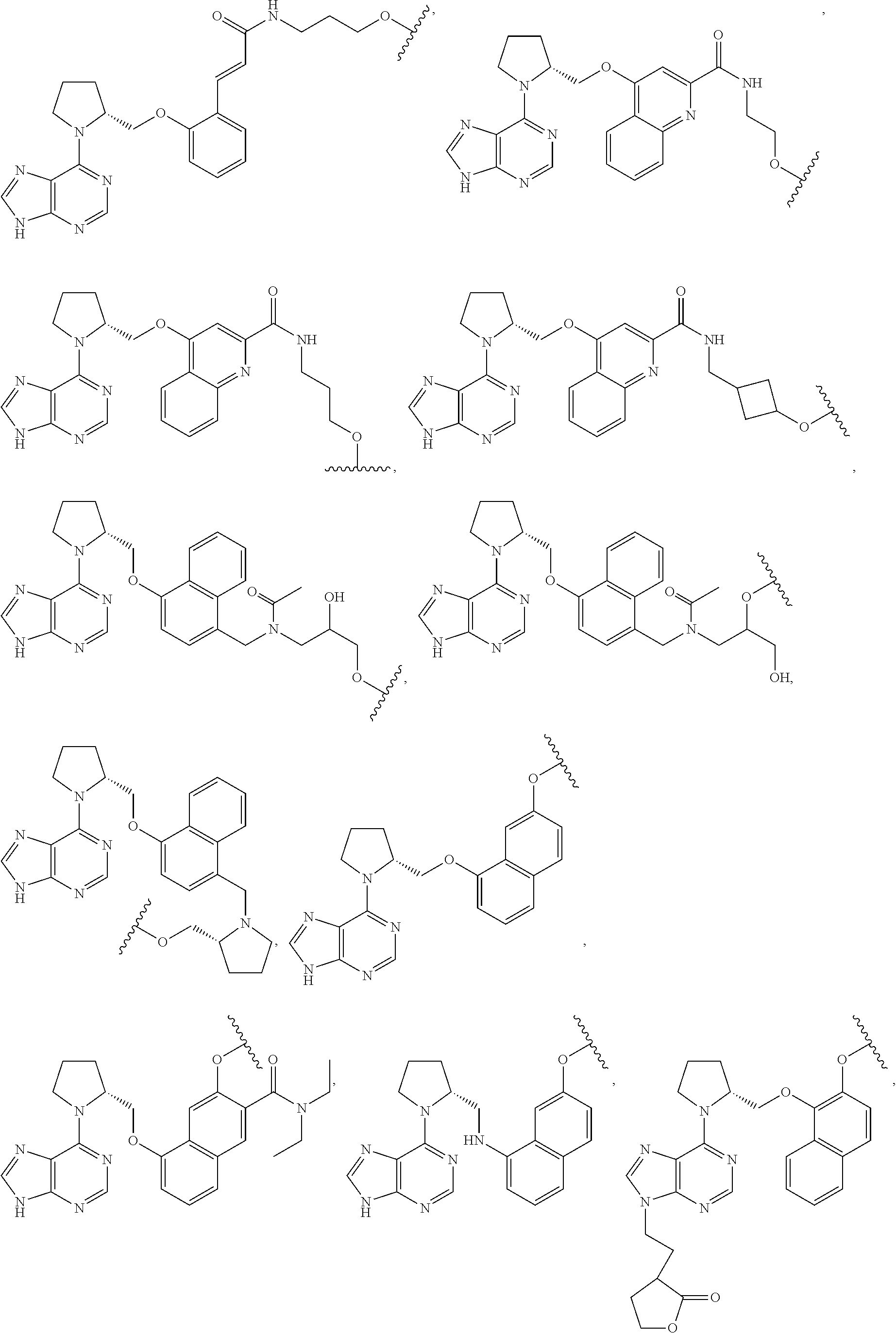

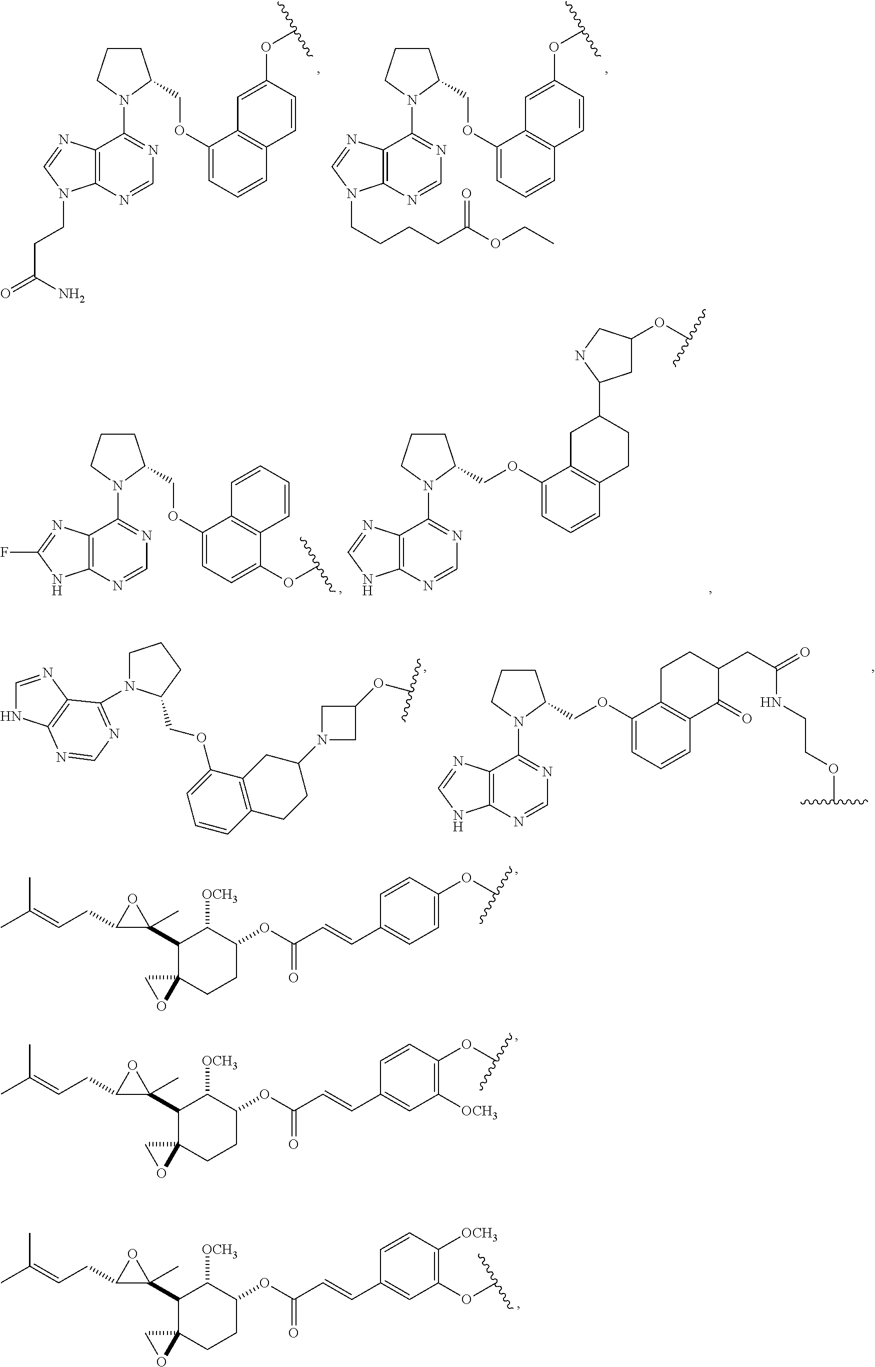

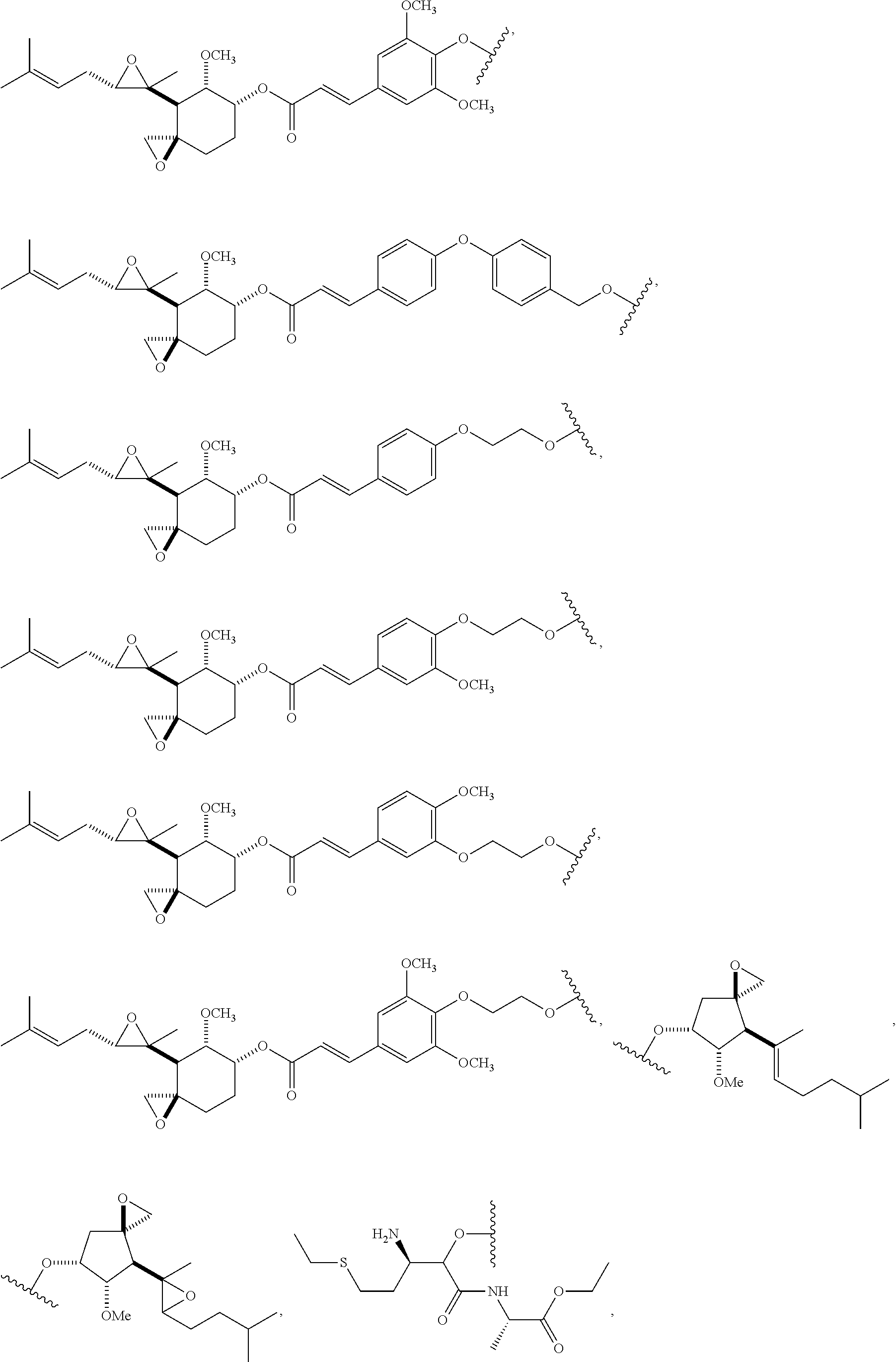

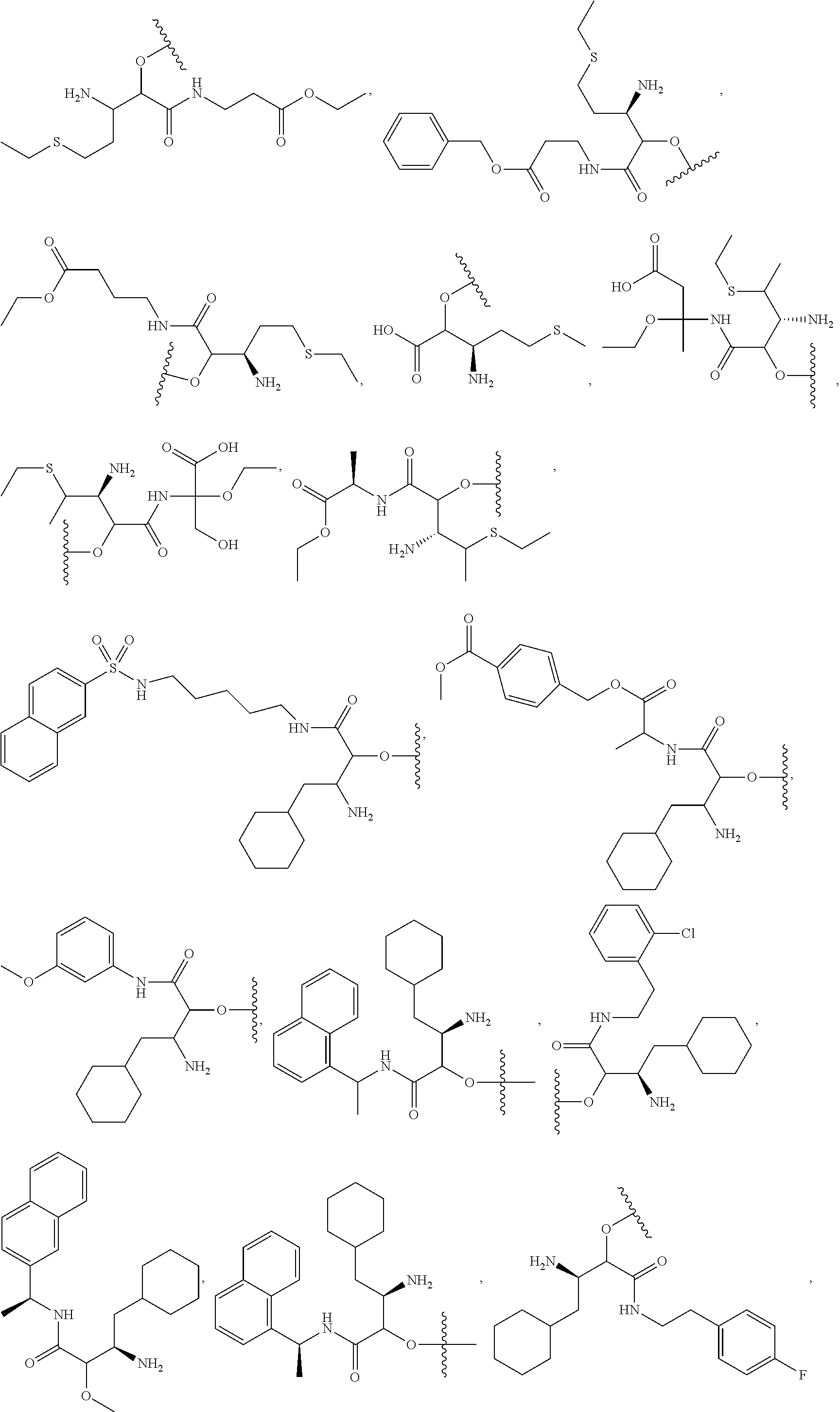

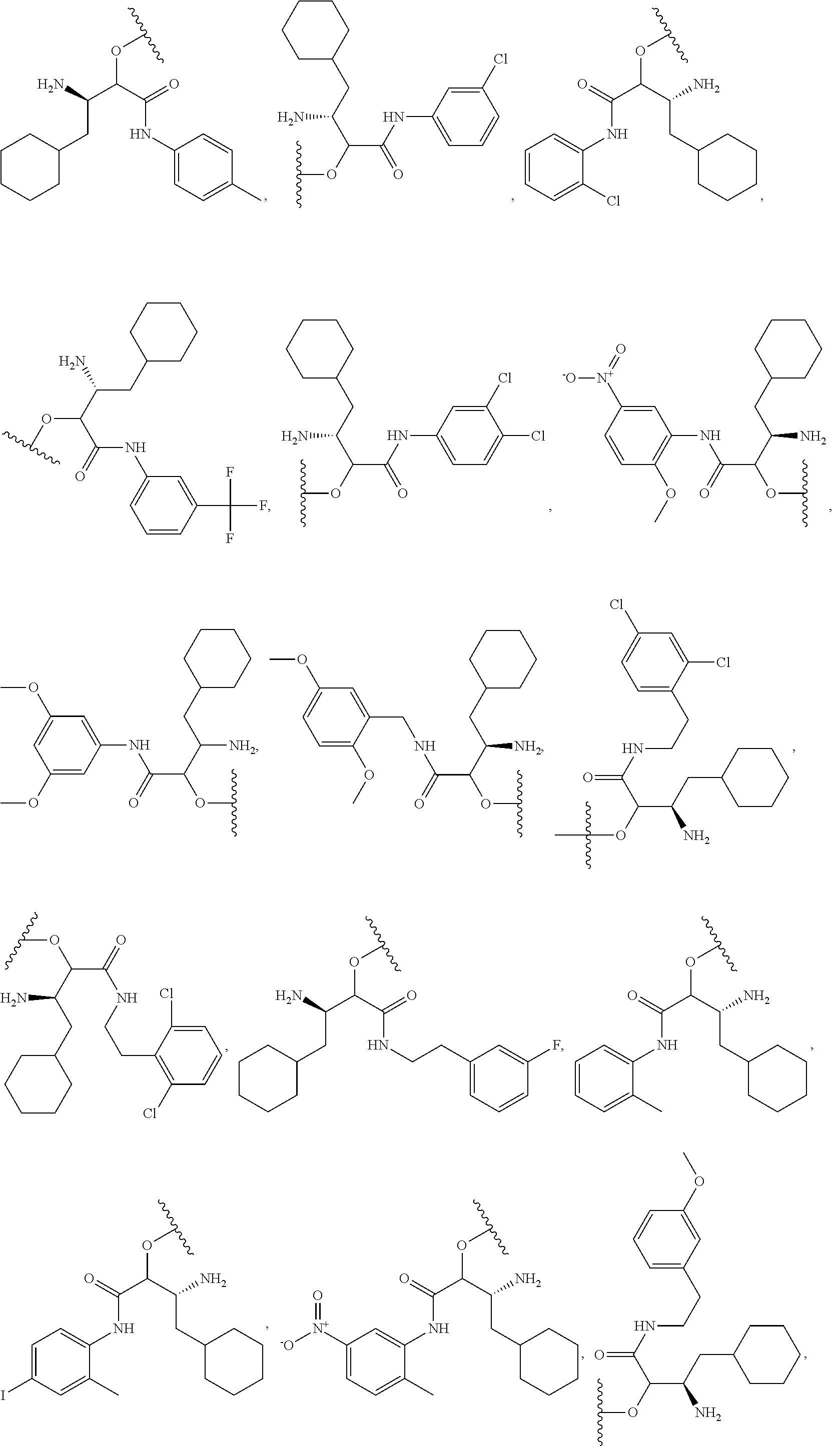

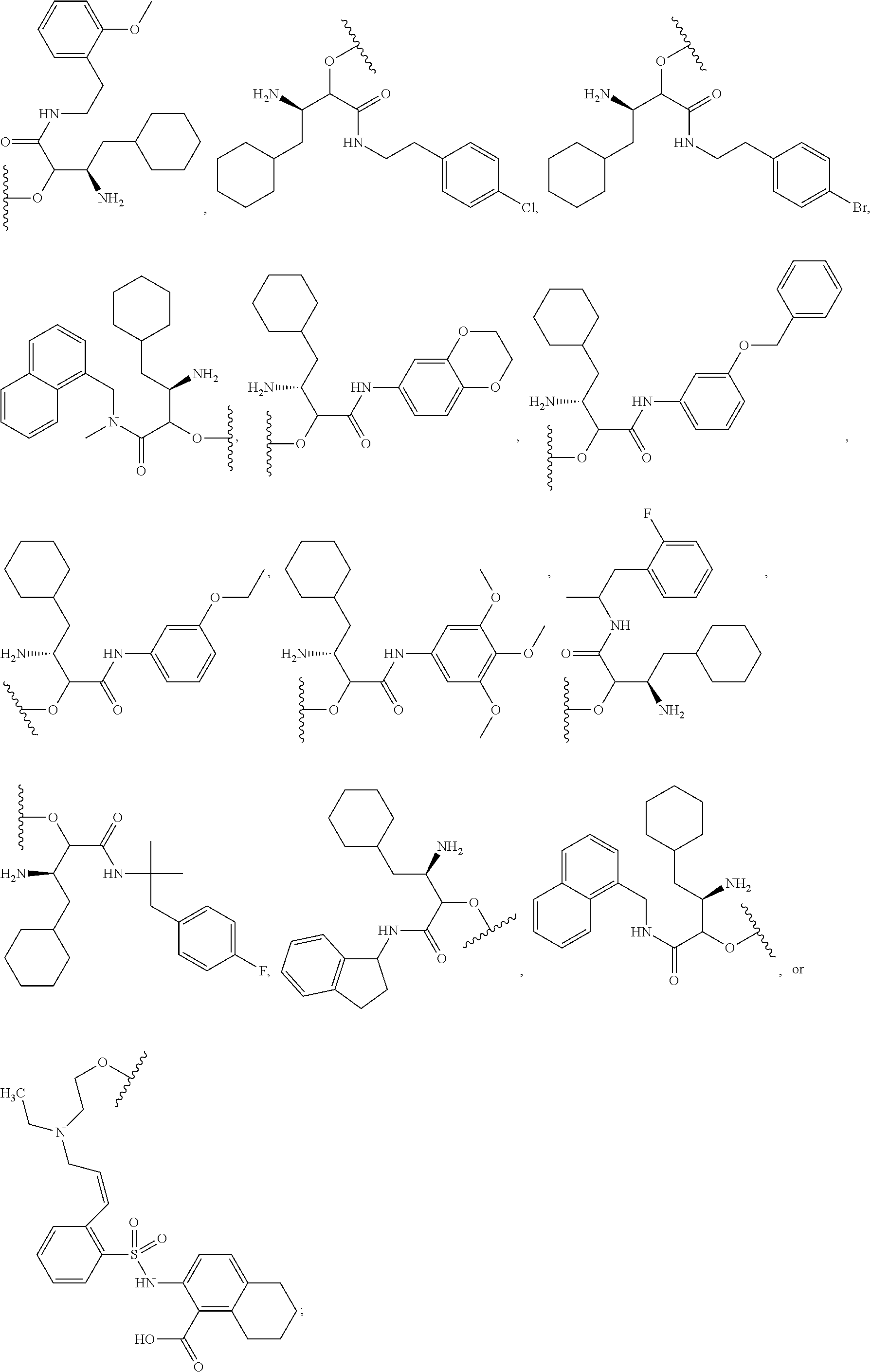

wherein R₂ is —OH or methoxy; and R₃ is H, —OH or methoxy.

In certain embodiments, W is

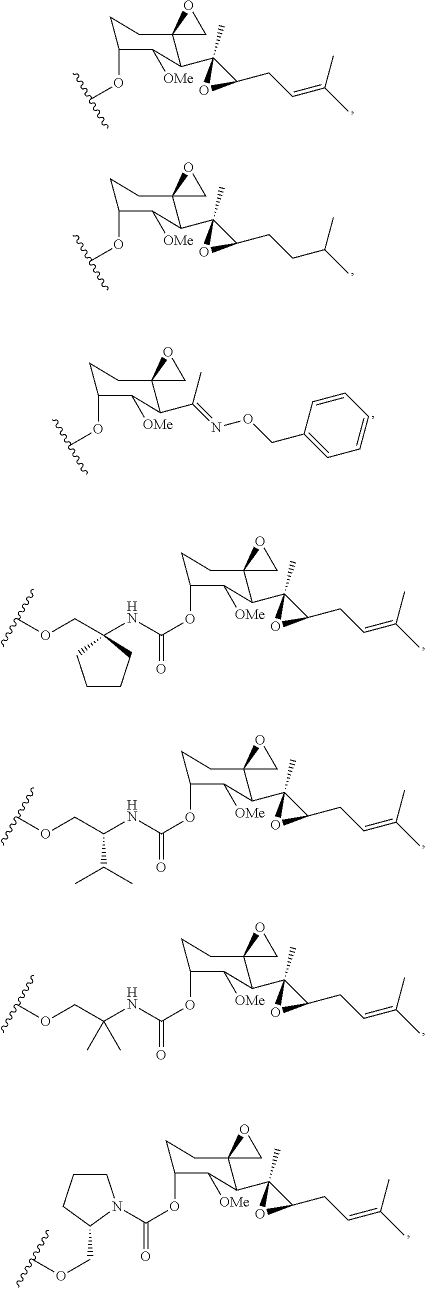

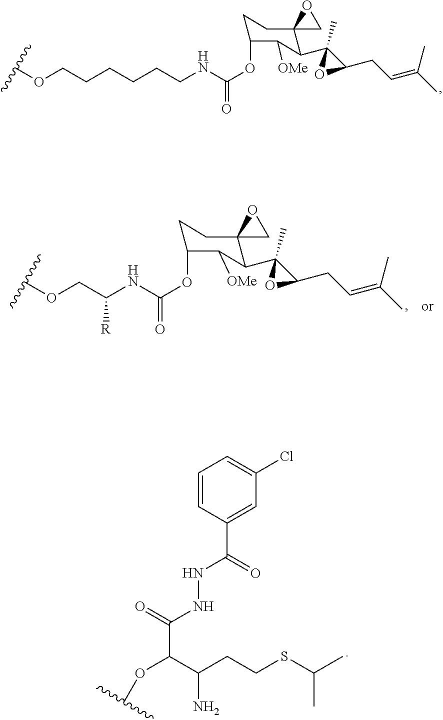

In certain embodiments, W is

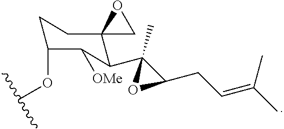

In certain embodiments, -Q-X—Y— is

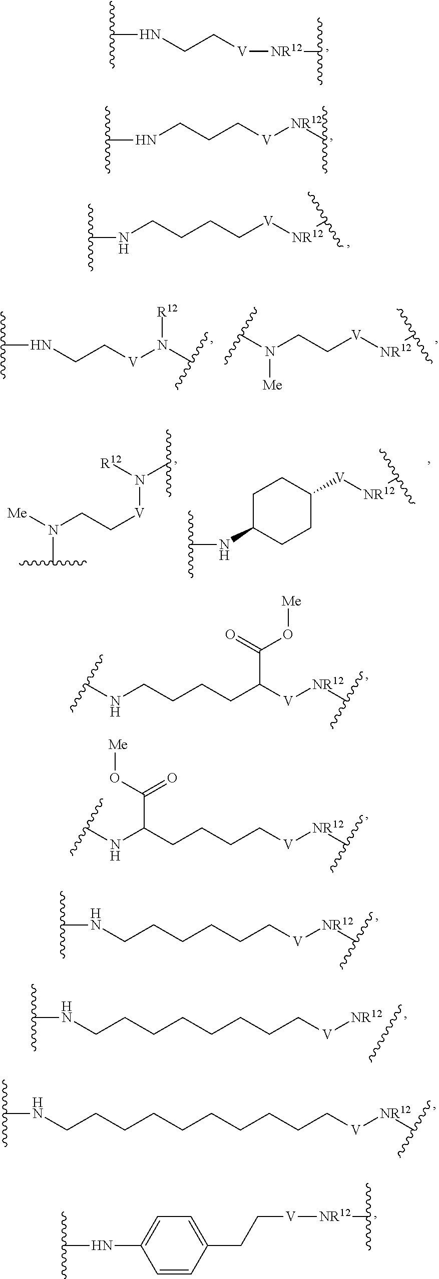

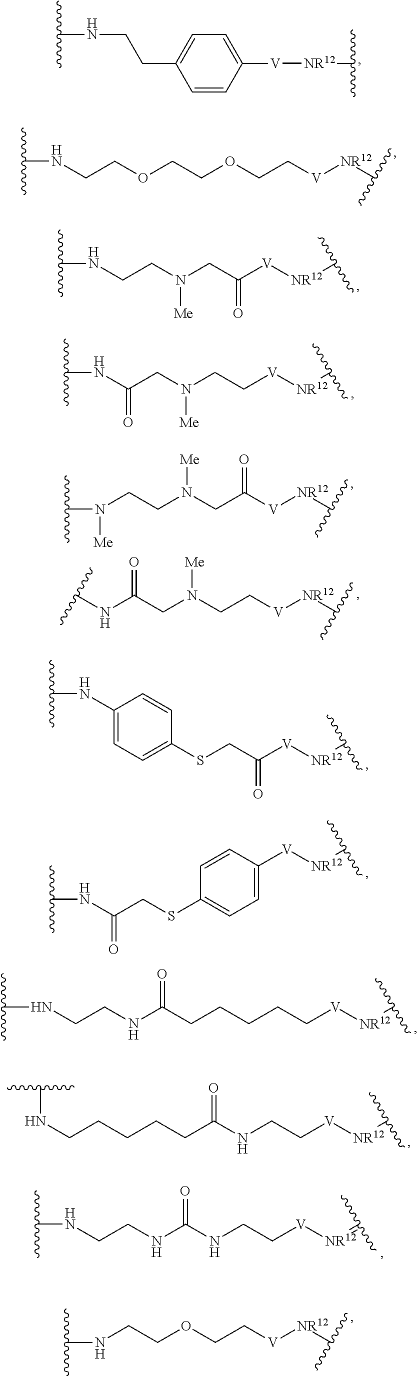

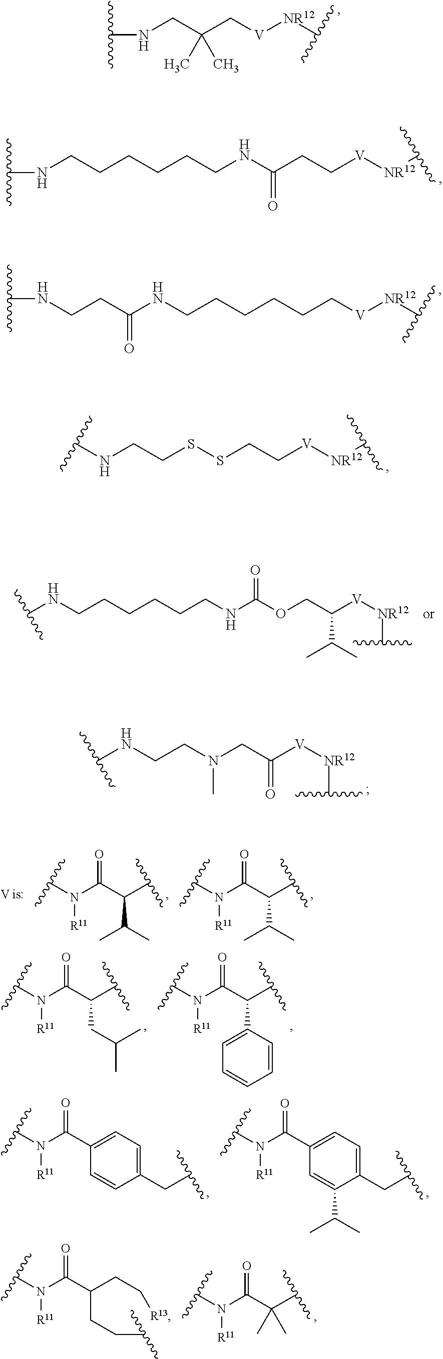

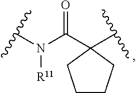

or a bond; R¹² is H or Me; or R¹² taken together with R¹⁴ forms a piperidine ring; R¹¹ is H or Me; and R¹³ taken together with R¹² forms a piperidine ring.

In certain embodiments, Z is H₂N-AA₆-C(O)—; AA₆ is glycine; Q-X—Y is

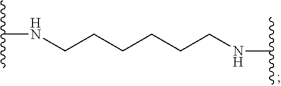

### and W is

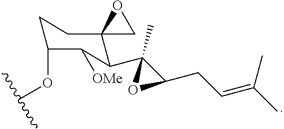

In certain embodiments, Z is H; Q-X—Y is

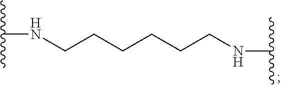

### and W is

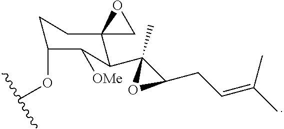

In certain embodiments, Z is H₂N-AA₆-C(O)—; AA₆ is glycine; Q-X—Y is

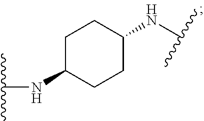

### and W is

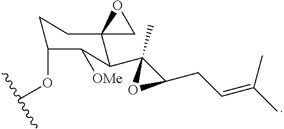

In certain embodiments, Z is H; Q-X—Y is

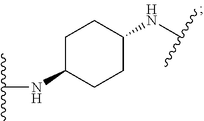

### and W is

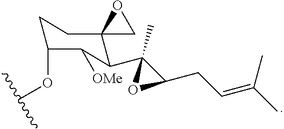

In certain embodiments, Z is H₂N-AA₆-C(O)—; AA₆ is glycine; Q-X—Y is

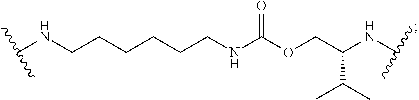

### and W is

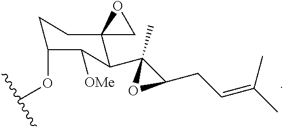

In certain embodiments, Z is H; Q-X—Y is

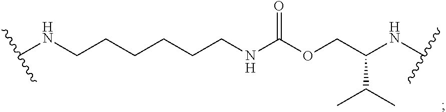

### and W is

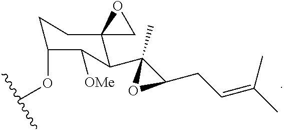

Other active moieties that may be modified to be used in conjugates of the invention include the following structures:

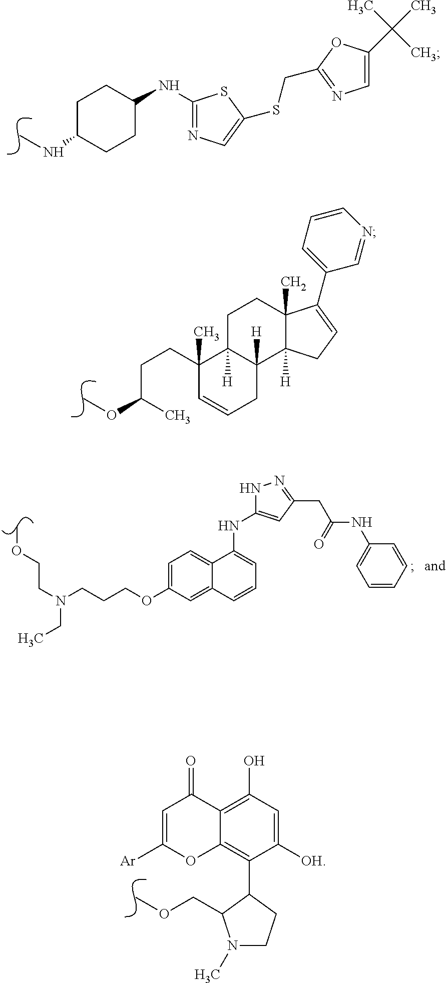

For purposes of this invention, the chemical elements are identified in accordance with the Periodic Table of the Elements, CAS version, Handbook of Chemistry and Physics, 67th Ed., 1986-87, inside cover.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. If, for instance, a particular enantiomer of a compound of the present invention is desired, it may be prepared by asymmetric synthesis or by derivation with a chiral auxiliary, where the resulting diastereomeric mixture is separated and the auxiliary group cleaved to provide the pure desired enantiomer. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomer.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

Examples below show that polymer conjugates of fumagillin analogs are more effective than the small molecule at doses below 2 mole % of the parent drug. Without being limited by any particular theory or mechanism of action, it is believed that the difference relates to significant intracellular target inhibition prior to active small molecule efflux. Examples below show that coupling a novel derivative of a fumagillol core provides efficacy at very low doses relative to the small molecule under conditions that reduce the plasma AUC of the active metabolite. The reduction in plasma AUC of the active metabolite results in a reduced systemic exposure to drug which reduces toxicity and increases safety. In the case of SDX-7320, the reduction in active dose is at least 10 fold (B16 model) and has been as much as 50 fold (A549 model).

### General Procedures

Tangential Flow Filtration (TFF) was used to purify the polymer products of the invention. TFF was performed with a Pall Minimate™ Capsule and Minimate™ TFF system according to the manufacturer's instructions. Either a Minimate TFF Capsule with 5 kDa Omega membrane (5K) or Minimate TFF Capsule with 10 kDa Omega membrane (10K) cartridge was used for purification. In all cases, the permeate was discarded and the retentate lyophilized to yield the polymer product. Structures of products were confirmed by ¹H NMR, small molecules were also characterized by MS. Polymer weights reported in the examples were not corrected for water content.

Carbamoylfumagillol and chloroacetylcarbamoylfumagillol can be prepared according to the methods disclosed in U.S. Pat. No. 5,166,172 (Kishimoto, et al., incorporated herein by reference). p-Nitrophenyl fumagill-6-yl carbonate can be prepared according to published procedures. (See Han, C. et al. Biorg. Med. Chem. Lett. 2000, 10, 39-43). MA-GFLG-ONp can be prepared according to the methods disclosed in U.S. Pat. No. 5,258,453 (Kopecek et al. incorporated herein by reference.)

### Example 1

### Synthesis of poly(HPMA-co-MA-GFLG-ONp)

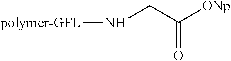

A mixture of hydroxypropylmethacrylamide (HPMA, 22.16 g, 155 mmol), N-methyacryl-gly-phe-leu-gly p-nitrophenyl ester (MA-GFLG-ONp, 10.00 g, 17.19 mmol), AIBN (1.484 g, 9.037 mmol) and acetone (225 g) was degassed (freeze, pump, thaw, 4 cycles). The resulting reaction mixture was stirred at 50° C. for 48 hours, then cooled to room temperature. The desired product was purified by trituration with acetone, then dried under vacuum to yield 17.6 g of poly(HPMA-co-MA-GFLG-ONp) as a white solid. The structure was verified by ¹H NMR and the product shown to be free from substantial impurities (e.g., p-nitrophenol). Based on UV absorbance, the copolymer contained 0.47 mmoles of p-nitrophenyl ester per gram of polymer. The copolymer of this example is used in most of the subsequent examples. A wide range of copolymers based on different monomers and/or monomer ratios may be made following this procedure by adjusting the stoichiometry and/or using different monomers.

### Example 2

### Synthesis of poly(HPMA-co-MA-GFLG-OH)

Poly(HPMA-co-MA-GFLG-ONp) (700 mg) was added portionwise to a solution of 0.1 M NaOH (11.3 mL) at 0° C. The yellow reaction mixture was stirred at 0° C. for 0.5 hours, then at room temperature for 4 hours. One-half of the solution was acidified with 0.1 M HCl to pH=6. The aqueous phase was extracted with ethyl acetate to remove excess p-nitrophenol. The aqueous phase was lyophilized to afford poly(HPMA-co-MA-GFLG-OH) as a colorless solid (360 mg).

### Example 3

### Synthesis of poly(HPMA-co-MA-GFLG-NHCH₂CH₂N(Me)BOC) and General Procedure A

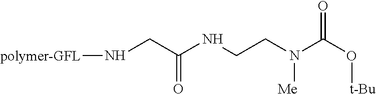

A solution of poly(HPMA-co-MA-GFLG-ONp) (1.0 g, 0.534 mmol) in DMF (6 mL) and H₂O (10 mL) was added dropwise over a 15 minute interval to a solution of tert-butyl N-(2-aminoethyl)-N-methylcarbamate (0.20 g, 1.15 mmol) in water (20 mL) at 0° C. The reaction mixture was stirred at 0° C. for 15 minutes, then warmed to room temperature and stirred for 12 hours. The solvents were evaporated under reduced pressure. The resulting residue was dissolved in water (50 mL), the pH was adjusted to approximately 8.0 with 0.1 M NaOH. The solution was filtered through a VacuCap filter, then purified using TFF (10 K). The polymer-containing solution was washed (as part of the TFF process) with 25 mM NaCl solution (800 mL) to remove p-nitrophenol, the pH of the solution was adjusted to approximately 4 with 0.1 M HCl, and then washed (as part of the TFF process) with water (400 mL). The polymer solution was lyophilized to isolate the compound poly(HPMA-co-MA-GFLG-NHCH₂CH₂N(Me)BOC) as a pale yellow solid (720 mg, 71%).

### Example 4

### Synthesis of poly(HPMA-co-MA-GFLG-NHCH₂CH₂NHMe)

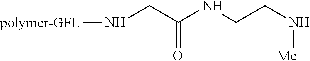

A solution of poly(HPMA-co-MA-GFLG-NHCH₂CH₂N(Me)BOC) (260 mg, 0.136 mmol) in D₂O (5.2 mL) was irradiated with microwave radiation at 150° C. with stirring for 6 hours. The ¹H NMR of this material indicated that deprotection of BOC group had occurred. The aqueous solution was lyophilized to isolate the poly(HPMA-co-MA-GFLG-NHCH₂CH₂NHMe) as a pale yellow solid (210 mg, 85%).

### Example 5

### Synthesis of N-({[2-(acetylamino)ethyl](methyl)amino}acetyl)carbamoylfumagillol and General Procedure B

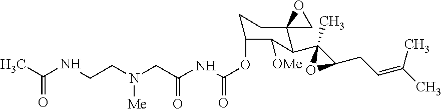

Diisopropylethylamine (DIEA) (130 mg) was added to a solution of N-[2-(methylamino)ethyl]acetamide hydrochloride (76 mg) and chloroacetylcarbamoylfumagillol (200 mg) in anhydrous DMF at 0° C. under N₂. The reaction mixture was allowed to warm to room temperature, and stirred for 12 hours. The solvent was removed under reduced pressure and the resulting residue was suspended in water (30 mL) and extracted with EtOAc (aqueous and organic phases from the emulsion formed were separated using a centrifuge) to remove excess chloroacetylcarbamoylfumagillol. Nitrogen was passed through the aqueous solution to reduce the residual level of EtOAc. The product was purified by flash chromatography (methanol/methylene chloride) to yield N-({[2-(acetylamino)ethyl](methyl)amino}acetyl)carbamoylfumagillol (75 mg) as an off-white foam.

### Example 6

### BocNHCH₂CH₂N(Me)CH₂C(O)NHC(O)₂-fumagill-6-yl (Alkylation of N-BOC, N′-methylethylenediamine with chloroacetylcarbamoylfumagillol)

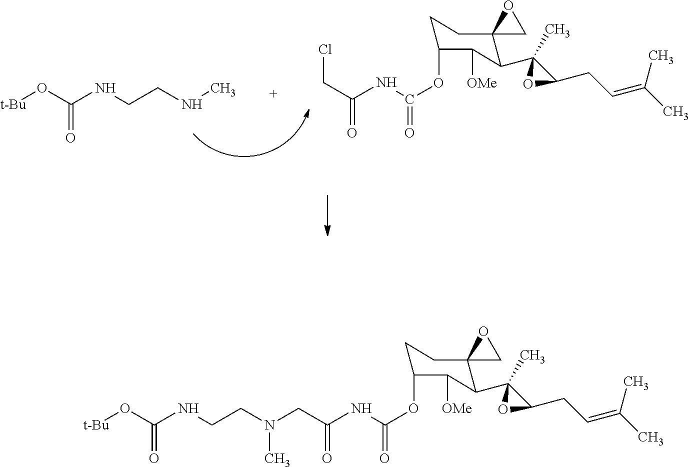

A solution of TNP-470 (0.2 g) and DIEA (0.105 g) in DMF (3 mL) was cooled to 0° C. A solution of tert-butyl N-[2-(methylamino)ethyl]carbamate (0.105 g) in DMF (3 mL) was added, and the mixture was stirred for 3 hours at 0° C. and then overnight. The reaction was diluted with ethyl acetate and extracted with water. The aqueous phase was back extracted with ethyl acetate, and the combined organic phases were extracted with brine, dried (MgSO₄) and evaporated to afford an oil. Purification by silica gel chromatography (methanol/methylene chloride) and evaporation of the product fractions gave BocNHCH₂CH₂N(Me)CH₂C(O)NHC(O)₂-fumagill-6-yl a white foam (0.16 g, 60%).

### Example 7

### Reaction of tert-butyl N-[2-aminoethyl]carbamate with chloroacetylcarbamoylfumagillol

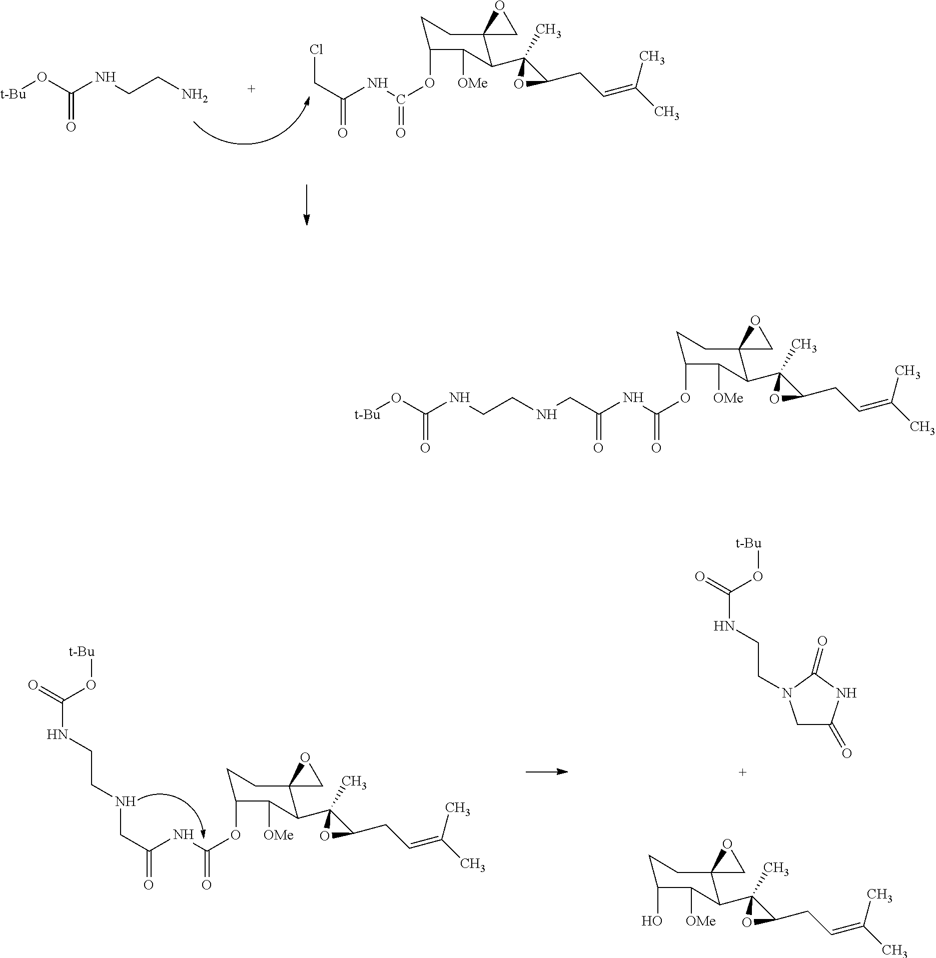

A 30 uL aliquot of a 1 M solution of Boc-ethylenediamine in DMF was added to DMF (270 uL). The solution was cooled to 0° C., and a solution of TNP-470 (48 mg) in DMF (600 uL) was added dropwise over 2 minutes. The reaction was monitored by LC/MS. The largest amount of the desired alkylation product observed was 34%. Carbamoylfumagillol was also produced. The ratio of desired product to carbamoylfumagillol was 1.0 to 0.4. Attempted isolation of the desired product resulted in the isolation of hydantoin and fumagillol. Thus, the desired product could not be isolated because of the rate of decomposition. Thus TNP-470 could not be alkylated according to the described method.

### Example 8

### Synthesis poly(HPMA-co-MA-GFLG-NHCH₂CH₂N(Me)CH₂C(O)NHC(O)₂-fumagill-6-yl)

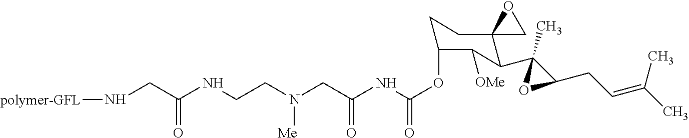

General Procedure B was followed using poly(HPMA-co-MA-GFLG-NHCH₂CH₂NHMe) (105 mg, 0.058 mmol) and chloroacetylcarbamoylfumagillol (46 mg, 0.114 mmol) in DMF (5 mL) to which DIEA (29.5 mg, 0.228 mmol) was added N₂. The product was purified using TFF (5 K) by washing with water (150 mL) to remove DIEA hydrochloride. The polymer solution was lyophilized to obtain the polymer conjugate (60 mg, 48%) as a pale yellow solid.

### Example 9

### Synthesis of poly(HPMA-co-MA-GFLG-NHCH₂CH₂NH₂.HCl) and General Procedure C for the Reaction of Diamines with poly(HPMA-co-MA-GFLG-ONp)

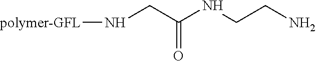

A solution of ethylenediamine (0.33 g, 5.49 mmole) in water (20 mL), pH 11.7, was adjusted to pH 9.1 by the addition of 37% aq HCl (17-18 drops). The solution was cooled in an ice bath and poly(HPMA-co-MA-GFLG-ONp) (1.03 g) in DMF (6 mL) was added dropwise over 20 minutes while maintaining the temperature below 4° C. The solution was stirred 20 minutes at 4° C., 50 minutes at room temperature to give a lemon yellow solution, pH 8.1. The solution was evaporated at 40° C. H₂O (3×10 mL) was added and evaporated. The product was diluted with water (60 mL), the solution adjusted with NaOH to pH 8.0. The solution was filtered through a VacuCap filter and purified by TFF as follows. The polymer solution was first washed with 25 mM NaCl solution (800 mL) to remove p-nitrophenol. The solution was washed with water (400 mL) then adjusted to pH 4 with 0.1 M HCl. The TFF retentate was collected and the filter was washed with 2×10 mL of water. The combined retentate and washes gave a polymer solution which was lyophilized to isolate the compound poly(HPMA-co-MA-GFLG-NHCH₂CH₂NH₂.HCl) as a pale yellow solid (0.71 g, 72%).

### Example 10

### Synthesis of poly(HPMA-co-MA-GFLG-N(Me)CH₂CH₂NHMe.HCl)

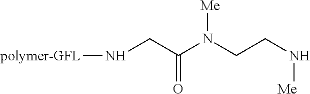

(free base shown)

General Procedure C was followed using N,N′-dimethylethylenediamine (0.47 g, 5.36 mmol) and poly(HPMA-co-MA-GFLG-ONp) (1.0 g) to yield poly(HPMA-co-MA-GFLG-N(Me)CH₂CH₂NHMe.HCl) as an off-white solid (0.78 g).

### Example 11

### Synthesis of poly(HPMA-co-MA-GFLG-N(Me)CH₂CH₂N(Me)CH₂C(O)NHC(O)₂-fumagill-6-yl)

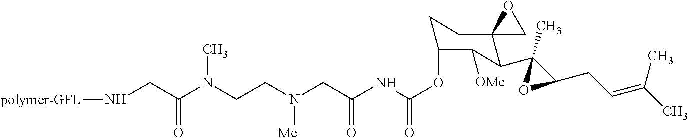

General procedure B was followed using poly(HPMA-co-MA-GFLG-N(Me)CH₂CH₂NHMe) (200 mg, 0.108 mmol) and chloroacetylcarbamoylfumagillol (86 mg, 0.213 mmol) to yield poly(HPMA-co-MA-GFLG-N(Me)CH₂CH₂N(Me)CH₂C(O)NHC(O)₂-fumagill-6-yl) as a pale yellow solid (180 mg).

### Example 12

### Synthesis of N-[(2R)1-hydroxy-2-methylbutan-2-yl]carbamoylfumagillol and General Procedure D

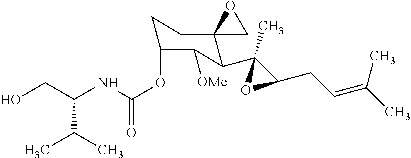

A solution of p-nitrophenyl fumagill-6-yl carbonate (400 mg, 0.89 mmol) and (R)-2-amino-3-methyl-l-butanol (280 mg, 2.71 mmol) were stirred in ethanol (10 mL) at room temperature for 12 hours. The yellow solution was concentrated and the residue purified by flash chromatography (methanol/methylene chloride) to yield N-[(2R)1-hydroxy-2-methylbutan-2-yl]carbamoylfumagillol (340 mg, 0.83 mmol) as a colorless oil.

### Example 13

### Synthesis of N-(6-hydroxyhexyl)carbamoylfumagillol

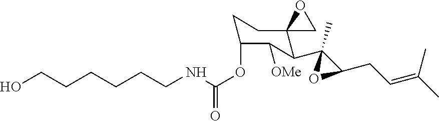

General Procedure D was followed using p-nitrophenyl fumagill-6-yl carbonate (150 mg) in ethanol (10 mL) and 6-aminohexanol (48 mg). The product was isolated as a colorless oil (110 mg, 78%).

### Example 14

### Synthesis of N-[1-(hydroxymethyl)cyclopentyl]carbamoylfumagillol

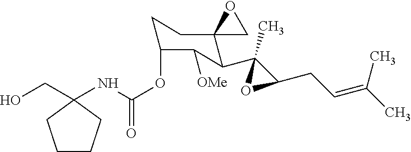

General Procedure D was followed using p-nitrophenyl fumagill-6-yl carbonate (100 mg) in ethanol (3 mL) and THF (1 mL) and cycloleucinol (52 mg) to afford N-[1-(hydroxymethyl)cyclopentyl]carbamoylfumagillol as an oil (50 mg).

### Example 15

### Synthesis of N-(1-hydroxy-2-methylpropan-2-yl)carbamoylfumagillol

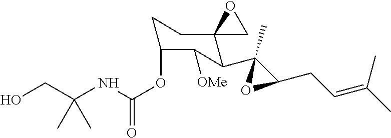

General Procedure D was followed using p-nitrophenyl fumagill-6-yl carbonate (100 mg) in ethanol (3 mL) and THF (2 mL) and 2-amino-2-methylpropanol (40 mg) to afford N-(1-hydroxy-2-methylpropan-2-yl)carbamoylfumagillol as an oil (37 mg).

### Example 16

### Synthesis of fumagill-6-yl (2S)-2-(hydroxymethyl)pyrrolidine-1-carboxylate

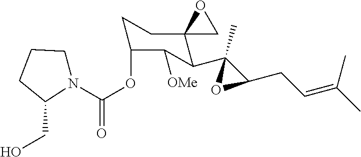

General procedure D was followed. The S-prolinol (68 mg, 0.67 mmol) was reacted with p-nitrophenyl fumagill-6-yl carbonate (150 mg, 0.335 mmol) in ethanol (4 mL) The product was purified by flash chromatography (methanol/methylene chloride) to yield fumagill-6-yl (2S)-2-(hydroxymethyl)pyrrolidine-1-carboxylate as a white foam (81 mg, 63%).

### Example 17

### Synthesis of fumagill-6-yl (2S)-2-({[(chloroacetyl)carbamoyl]oxy}methyl)pyrrolidine-1-carboxylate

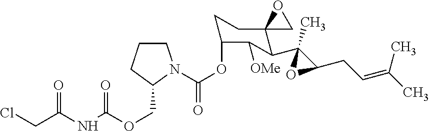

A solution of fumagill-6-yl (2S)-2-(hydroxymethyl)pyrrolidine-1-carboxylate (330 mg) in methylene chloride (2.1 mL) was cooled to 0° C. and chloroacetylisocyanate (115 mg) in methylene chloride (1.5 mL) was added dropwise. After 40 minutes, the mixture was diluted with methylene chloride (20 mL) and the organic phase washed with water (3×). The organic phase was dried (Na₂SO₄) and evaporated to yield fumagill-6-yl (2S)-2-({[(chloroacetyl)carbamoyl]oxy}methyl)pyrrolidine-1-carboxylate as a white foam (400 mg).

### Example 18

### Synthesis of poly[HPMA-co-MA-GFLG-NCH₂CH₂N(Me)-acetylcarbamoyl-[(2R)-1-hydroxy-3-methylbutan-2-yl]carbamoylfumagillol]

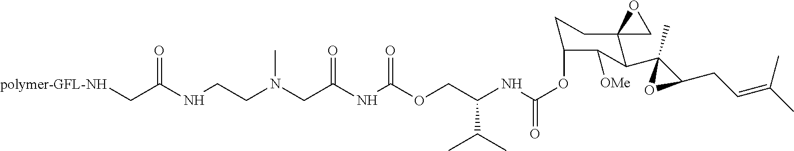

General procedure B was followed using chloroacetylcarbamoyl[(2R)-1-hydroxy-3-methylbutan-2-yl]carbamoylfumagillol (120 mg) (and poly(HPMA-co-MA-GFLG-NHCH₂CH₂NHMe) (200 mg) with DIEA (57 mg) in DMF (5 mL) to yield 2-poly[HPMA-co-MA-GFLG-NCH₂CH₂N(Me)]-acetylcarbamoyl-[1-hydroxy-3-methylbutan-2-yl]carbamoylfumagillol (200 mg, 80%).

### Example 19

### Synthesis of fumagill-6-yl 2-(poly[HPMA-co-MA-GFLG-NCH₂CH₂N(Me)]-acetylcarbamoylhydroxymethyl)pyrrolidine-1-carboxylate)

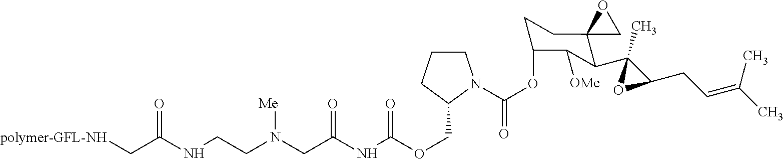

General procedure B was followed using the fumagill-6-yl (2S)-2-(chloroacetylcarbamoylhydroxymethyl)pyrrolidine-1-carboxylate (90 mg) (and poly(HPMA-co-MA-GFLG-NHCH₂CH₂NHMe) (200 mg) with DIEA (57 mg) in DMF (5 mL) to yield fumagill-6-yl2-poly[HPMA-co-MA-GFLG-NCH₂CH₂N(Me)]-acetylcarbamoylhydroxymethyl)pyrrolidine-1-carboxylate as a pale yellow solid (150 mg, 60%).

### Example 20

### Synthesis of N-(6-aminohexyl)carbamoylfumagillol

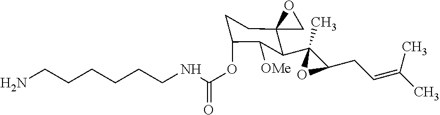

A solution of 1,6-diaminohexane (0.13 g) in methanol (8 mL) was cooled to 0° C. and p-nitrophenyl fumagill-6-yl carbonate (0.13 g) in methanol (2 mL) was added dropwise. The solvent was reduced to about 2 mL by rotary evaporation. Ethyl acetate was added and the organic phase was washed with water, 0.1 N NaOH, water, brine and dried with sodium sulfate. The solvent was evaporated and the residue dissolved in ethanol (15 mL). DL-tartaric acid (16 mg) was added, the solution was stored overnight and then evaporated to about 0.5 mL. Ether was added and a white solid formed. The solid was collected by filtration, washed with ether and dried to yield the tartrate salt of N-(6-aminohexyl)carbamoylfumagillol (74 mg).

### Example 21

### Synthesis of poly[HPMA-co-MA-GFLG-NH(CH₂)₆NH₂.HCl]

General Procedure C was followed using 1,6-diaminohexane (621 mg, 5.36 mmol) and poly(HPMA-co-MA-GFLG-ONp) (1.0 g). The crude product was purified by TFF (5 K) using aqueous NaCl (25 mM) and then acidified to pH 4.0 with 0.1 M HCl and further purified by TFF with water to yield poly[HPMA-co-MA-GFLG-NH(CH₂)₆NH₂.HCl] as an off-white solid (860 mg).

### Example 22

### Synthesis of p-nitrophenyl N-[(2R)1-hydroxy-2-methylbutan-2-yl]carbamoylfumagill-6-yl carbonate and General Procedure E

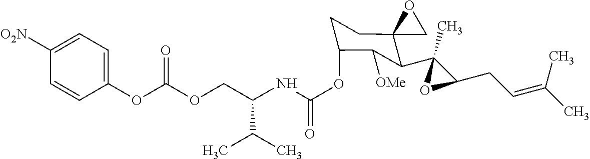

To a solution of the alcohol N-[(2R)1-hydroxy-2-methylbutan-2-yl]carbamoylfumagillol (1.11 g) in methylene chloride at 0° C. under N₂ was added DMAP (660 mg, 5.40 mmol) followed by the portionwise addition of p-nitrophenyl chloroformate (810 mg). The reaction mixture was stirred at 0° C. for 1 hour. The solvent was evaporated and the resulting residue was dissolved in EtOAc and washed with water, brine and dried (Na₂SO₄). Evaporation of EtOAc provided the crude product, which was purified by flash chromatography (silica, eluting with 100% hexanes and then with 2-30% EtOAc). The fractions containing pure product were combined and evaporated to isolate N-[(2R)1-(p-nitrophenolcarbonylhydroxy-2-methylbutan-2-yl]carbamoylfumagillol (1.25 g, 80%) as a white solid.

### Example 23

### Synthesis of N-[1-(p-nitrophenoxycarbonylhydroxymethyl)-2-methylpropan-2-yl)carbamoylfumagillol

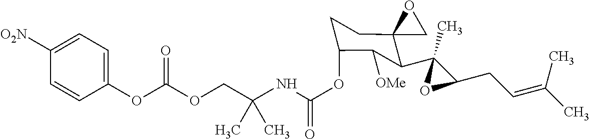

Following General Procedure E, dimethylalcohol (60 mg), p-nitrophenyl fumagill-6-yl carbonate (46 mg), and DMAP (37 mg) were reacted in methylene chloride (8 mL). The reaction mixture was diluted with ethyl acetate and washed with water (3×) and then brine. The organic phase was dried (Na₂SO₄) and evaporated to a yellow foam (87 mg) which was used without further purification.

### Example 24

### Synthesis of N-[1-(p-nitrophenoxycarbonylhydroxymethyl)cyclopentyl]carbamoylfumagillol

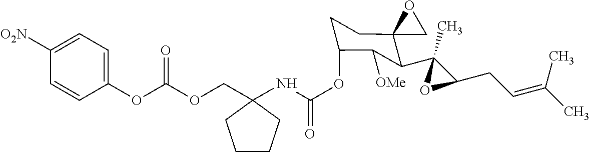

Following General Procedure E, N-[1-(hydroxymethyl)cyclopentyl]carbamoylfumagillol (product from Example 14, 74 mg), p-nitrophenyl chloroformate (53 mg), and DMAP (43 mg) were reacted in methylene chloride (5 mL). After the extractive workup, N-[1-(p-nitrophenoxycarbonylhydroxymethyl)cyclopentyl]carbamoylfumagillol (100 mg) was used without further purification.

### Example 25

### Synthesis of poly[HPMA-co-MA-GFLG-NH(CH₂)₆NHcarbamoyl-[1-hydroxy-3-methylbutan-2-yl]carbamoylfumagillol] and General Procedure F

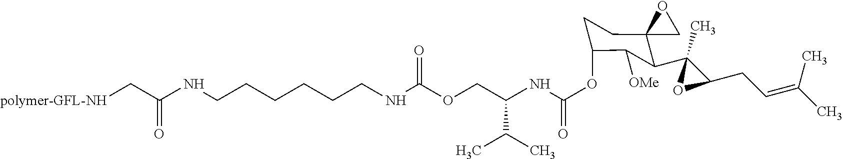

To a solution of polymer (400 mg) and p-nitrophenyl N-[(2R)1-hydroxy-3-methylbutan-2-yllcarbamoylfumagill-6-yl carbonate (240 mg) in DMF (8 mL) at 0° C. was added DIEA (0.11 g) dropwise. The solution was stirred at 0° C. for one hour and allowed to warm to room temperature. After 3 days, the solvent was evaporated and water (80 mL) was added. The aqueous phase was extracted with ethyl acetate (500 mL total) until none of the starting carbonate was detectable by MS. The aqueous phase was purified by TFF (10 K) and the retentate lyophilized to yield the conjugate as a white solid (380 mg, 77%).

¹H NMR (DMSO-d6): δ 8.25 (bs, 2H, amide-NH), 8.0 (bs, 1H, amide-NH), 7.70 (bs, 2H, amide-NH), 7.10-7.30 (m, 15H, Phenylalanine and amide-NH), 7.10 (bt, 1H, NH-Fum), 6.92 (bd, 1H, NH-Fum), 5.26 (m, H-5-Fum), 5.18 (bt, alkene-Fum), 4.50-4.80 (m, 1H, phenylalanine alpha proton), 4.0-4.21 (m, 1H, leucine alpha proton), 3.50-3.84 (m, 19H), 3.29 (s, 3H, OMe-Fum), 2.80-3.10 (m, 28H), 2.51 (d, 1H, J=4.4 Hz, H-2-Fum), 2.19 (m, 2H, allylic-Fum), 0.82-1.92 [m 131H {1.84 (m, 2H, Fum), 1.72 (s, 3H, Fum-Me), 1.60 (s, 3H, Fum-Me), 1.09 (s, 3H, Fum-Me), 0.84 (dd, 6H, Fum-isopropyl}].

### Example 26

### Synthesis of poly[HPMA-co-MA-GFLG-N-(2-aminoethyl)carbamoylfumagillol]

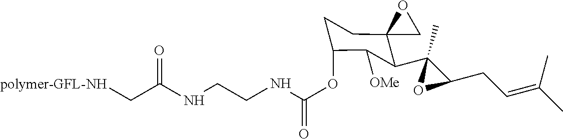

General procedure F was followed using poly(HPMA-co-MA-GFLG-NHCH₂CH₂NH₂.HCl) (200 mg), p-nitrophenyl fumagill-6-yl carbonate (100 mg) and DIEA (57 mg) in DMF (10 mL). The product was purified by TFF (10 K) with water and lyophilized to yield the conjugate as a pale yellow solid (160 mg).

### Example 27

### Synthesis of poly[HPMA-co-MA-GFLG-N(Me)-(2-methylaminoethyl)carbamoylfumagillol]

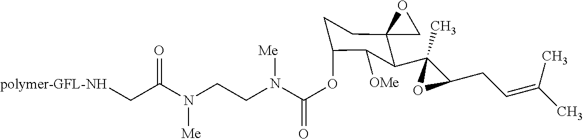

General procedure F was followed using poly(HPMA-co-MA-GFLG-N(Me)CH₂CH₂NHMe.HCl) (200 mg), p-nitrophenyl fumagill-6-yl carbonate (100 mg) and DIEA (57 mg) in DMF (5 mL). The product was purified using TFF (10 K) with water and lyophilized to yield the conjugate as an off-white solid (180 mg).

### Example 28

### Synthesis of poly(HPMA-co-MA-GFLG-N-(2-aminoethyl)carbamoyldihydrofumagillol

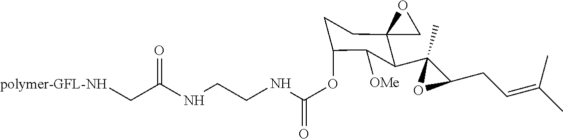

General procedure F was followed using poly(HPMA-co-MA-GFLG-NHCH₂CH₂NH₂.HCl) (200 mg), p-nitrophenyl dihydrofumagill-6-yl carbonate (200 mg) and DIEA (57 mg) in DMF (10 mL). The product was purified by TFF (10 K) with water (150 mL) and lyophilized to yield poly(HPMA-co-MA-GFLG-N-(2-aminoethyl)carbamoyldihydrofumagillol as a pale yellow solid (160 mg).

### Example 29

### Synthesis of poly[HPMA-co-MA-GFLG-N-(3-aminopropyl)carbamoylfumagillol]

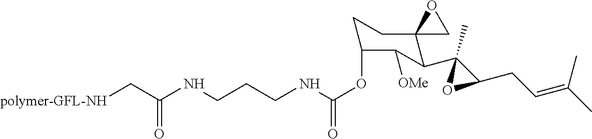

General procedure F was followed using poly(HPMA-co-MA-GFLG-NHCH₂CH₂ CH₂NH₂.HCl) (220 mg), p-nitrophenyl fumagill-6-yl carbonate (110 mg) and DIEA (63 mg) in DMF (6 mL). The solvent was evaporated and the resulting solution diluted with water. The aqueous phase was extracted with ethyl acetate and purified by TFF using 350 mL of water. The retentate was lyophilized to yield poly[HPMA-co-MA-GFLG-N-(3-aminopropyl)carbamoylfumagillol] as a light pink powder (200 mg).

### Example 30

### Synthesis of poly[HPMA-co-MA-GFLG-N-(6-aminohexyl)carbamoylfumagillol]

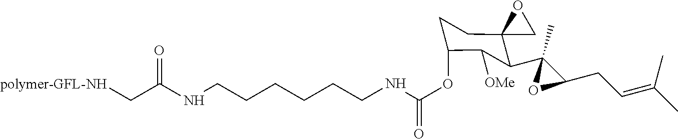

General procedure F was followed using poly[HPMA-co-MA-GFLG-N-(trans-4-aminocyclohexylamine.HCl)] (1.0 g), p-nitrophenyl fumagill-6-yl carbonate (0.48 g) and DIEA (0.27 g) in DMF (25 mL). The solvent was evaporated and the solution diluted with water. The aqueous phase (300 mL) was extracted with ethyl acetate (700 mL total) and purified by TFF using an additional 350 mL of water. The retentate was lyophilized to yield poly[HPMA-co-MA-GFLG-N-(4-aminocyclohexyl)carbamoylfumagillol] as a light pink solid (0.9 g).

¹H NMR (DMSO-d6): δ 8.10-8.35 (m, 3H, amide-NH), 7.90-8.10 (m, amide-NH), 7.05-7.32 (m, 22H, amide-NH) 5.27 (m, H-5-Fum), 5.18 (bt, alkene-Fum), 4.60-4.90 (m, 14H), 4.50-4.60 (m, 1H, phenylalanine alpha proton), 4.10-4.30 (m, 1H, leucine alpha proton), 3.40-3.80 (m, 21H), 3.27 (s, 3H, OMe-Fum), 2.80-3.20 (m, 33H), 2.56 (d, 1H, H=3.90 Hz, H-2-Fum), 2.18 (m, 2H, allylic-Fum), 0.37-2.0 [m, 147H {1.70 (s, 3H, Fum-Me), 1.60 (s, 3H, Fum-Me), 1.07 (s, 3H, Fum-Me){].

### Example 31

### Synthesis of poly[HPMA-co-MA-GFLG-N-(trans-4-aminocyclohexyl)carbamoylfumagillol]

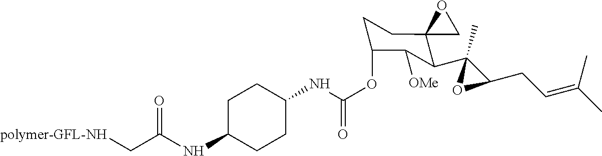

General procedure F was followed using poly[HPMA-co-MA-GFLG-N-(trans-4-aminocyclohexylamine.HCl)](1.0 g), p-nitrophenyl fumagill-6-yl carbonate (0.48 g) and DIEA (0.27 g) in DMF 25 mL. The solvent was evaporated and the solution diluted with water. The aqueous phase (300 mL) was extracted with ethyl acetate (700 mL total) and purified by TFF using an additional 350 mL of water. The retentate was lyophilized to yield poly[HPMA-co-MA-GFLG-N-(3-aminohexyl)carbamoylfumagillol] as a light pink solid (0.9 g).

¹H NMR (DMSO-d6): δ 7.90-8.35 (m, 4H, amide-NH), 7.0-7.70 (m, 25H, Phenylalanine and amide-NH), 5.26 (m, H-5-Fum), 5.18 (bt, alkene-Fum), 4.60-4.90 (m, 14H), 4.50-4.60 (m, 1H, phenylalanine alpha proton), 4.10-4.30 (m, 1H, leucine alpha proton), 3.40-3.80 (m, 21H), 3.26 (s, 3H, OMe-Fum), 2.80-3.10 (m, 31H), 2.17 (m, 2H, allylic-Fum), 0.37-2.0 [m, 166H {1.69 (s, 3H, Fum-Me), 1.59 (s, 3H, Fum-Me), 1.07 (s, 3H, Fum-Me)}].

### Example 32

### Synthesis of poly[HPMA-co-MA-GFLG-N-[2-(4-aminophenyl)ethyl]carbamoylfumagillol]

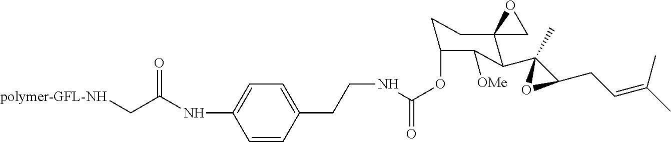

To a suspension of poly[HPMA-co-MA-GFLG-OH] (200 mg), N-[2-(4-aminophenyl)ethyl]carbamoylfumagillol](100 mg) and DIEA (75 mg) in DMF (6 mL) at 0° C. was added EDCI (total 44 mg) in portions. The solution was allowed to warm to room temperature and stirred overnight. The solvent was evaporated, the residue was suspended in water and the suspension extracted with EtOAc (7 times, total 250 mL). The aqueous phase was purified by TFF (10 K) using water (350 mL). The retentate was lyophilized to afford the polymer as a white fluffy solid (170 mg).

### Example 33

### Synthesis of poly[HPMA-co-MA-GFLG-NH-2-[(2-(2-aminoethoxy)ethoxy)ethyl]carbamoylfumagillol]

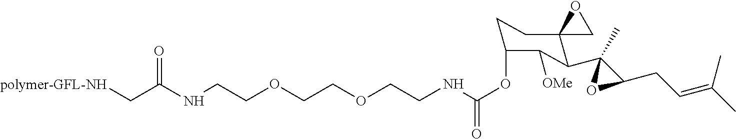

To a solution of 2,2′-(Ethylenedioxy)bis(ethylamine) (0.79 g, 5.34 mmol) in distilled water (20 mL) at 0° C. (pH=11.56) was added conc. HCl until pH of the solution was 9.01 (measured by pH meter). Poly(HPMA-co-MA-GFLG-ONp) (1.0 g, 0.534 mmol) in DMF (6 mL) and H₂O (10 mL) was added to the amine-containing solution dropwise over a period of 15 minutes and the reaction mixture was stirred at 0° C. for 15 minutes. The reaction mixture was then allowed to warm to room temperature and stirred for 2 hours. The pH of the solution was measured to be 8.15. The reaction mixture was diluted with distilled water (300 mL) and filtered through a VacuCap filter, reaction flask was washed with water (100 mL). The polymer solution was concentrated to 40 mL by TFF (10 K) and was washed with 25 mM NaCl (800 mL) to remove p-nitrophenol, the pH was then adjusted to 4 with 0.1 M HCl and finally washed with water (400 mL). The pure polymer solution was lyophilized to isolate poly[HPMA-co-MA-GFLG-NH-2-[2-(2-aminoethoxy)ethoxy]ethylamine.HCl] as a pink solid (800 mg, 78%).

To a mixture of p-nitrophenyl fumagill-6-yl carbonate (93 mg, 0.208 mmol) and poly[HPMA-co-MA-GFLG-N-2-[(2-(2-aminoethoxy)]ethoxy)ethylamine.HCl] (200 mg, 0.104 mmol) in anhydrous DMF (5 mL) at 0° C. under N₂ was added DIEA (57 mg, 0.416 mmol). The reaction mixture was allowed to warm to room temperature and stirred for 12 hours. The solvent was removed under reduced pressure and the resulting residue was suspended in water (30 mL) and extracted with EtOAc (aqueous and organic phases from the emulsion formed were separated using centrifuge) to remove excess of p-nitrophenyl fumagill-6-yl carbonate and p-nitrophenol. Nitrogen was passed through the aqueous solution to remove traces of EtOAc and it was purified using TFF (5K) by washing it with water (150 mL) to remove DIEA hydrochloride. The polymer solution was lyophilized to obtain the desired polymer conjugate poly[HPMA-co-MA-GFLG-N-2-[2-(2-aminoethoxy)ethoxyethyl]carbamoylfumagillol] (220 mg, 95%) as an off-white solid.

### Example 34

### Synthesis of poly[HPMA-co-MA-GFLG-NH-(6-aminodecyl)carbamoylfumagillol]

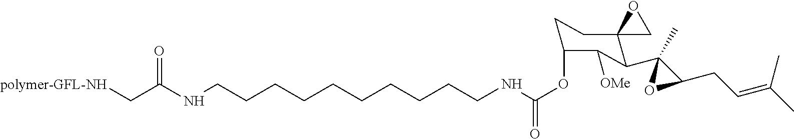

To a mixture of p-nitrophenyl fumagill-6-yl carbonate (300 mg, 0.67 mmol) and poly[HPMA-co-MA-GFLG-N-10-[decylamine.HCl] (300 mg, 0.15 mmol; made in a similar manner to Example 33 except 1,10-diaminodecane was used as the amine) in anhydrous DMF (6 mL) at 0° C. under N₂ was added DIEA (83 mg, 0.64 mmol). The reaction mixture was allowed to warm to room temperature and stirred for 12 hours. The solvent was removed under reduced pressure and the resulting residue was suspended in water (30 mL) and extracted with EtOAc (aqueous and organic phases from the emulsion formed were separated using a centrifuge) to remove excess of p-nitrophenyl fumagill-6-yl carbonate and p-nitrophenol. Nitrogen was passed through the aqueous solution to remove traces of EtOAc. The crude aqueous solution was purified using TFF (10K) by washing with water (150 mL) to remove DIEA hydrochloride. The polymer solution was lyophilized to obtain the desired polymer conjugate poly[HPMA-co-MA-GFLG-NH-(10-aminodecyl)carbamoylfumagillol] (300 mg, 87%) as an off-white solid.

### Example 35

### Synthesis of N-(2-acetamidoethyl)carbamoylfumagillol

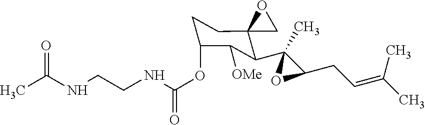

To a solution of p-nitrophenyl fumagill-6-yl carbonate (200 mg) in ethanol (5 mL) at 0° C. was added N-(2-aminoethyl)acetamide (0.132 mL).The solution was stirred at 0° C. for one hour and overnight at room temperature. The reaction was diluted with ethyl acetate, washed with water. The aqueous phase was back extracted with ethyl acetate and the combined organic phases dried (MgSO₄). The crude product was purified by flash chromatography. The product was a yellow solid (120 mg).

### Example 36

### Lysine Conjugate of Polymer and MetAP2Inhibitor Moiety

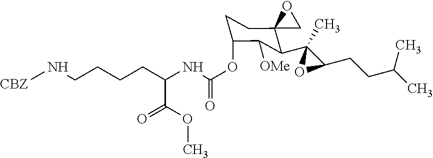

To a solution of p-nitrophenyl fumagill-6-yl carbonate (400 mg) and N-ε-Cbz-O-methyl-L-lysine hydrochloride in DMF (10 mL) at 0° C. was added DIEA (350 mg). The reaction was warmed to room temperature and the stirred overnight. The solution was diluted with ethyl acetate, washed with 0.1 N NaOH (4×), water, and then brine. The organic phase was dried (Na₂SO₄), filtered and evaporated. The residue was purified by flash chromatography (silica; methanol/methylene chloride) to provide the N-ε-Cbz-O-methyl-lysine-carbonylfumagillol (550 mg).

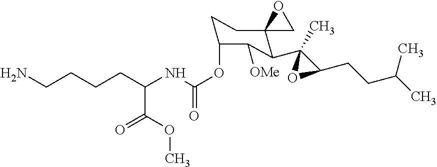

To a solution of N-ε-Cbz-O-methyl-lysine-carbonylfumagillol (200 mg) in ethyl acetate (10 mL) was added PtO₂ monohydrate (20 mg) and the solution hydrogenated at STP for 20 minutes. Reduction of the double bond but not deprotection of the Cbz was verified by MS. The solution was filtered and evaporated. The residue was dissolved in methanol (10 mL) and 10% Pd/C (20 mg) was added. The solution was hydrogenated under STP for 5 minutes, and removal of the Cbz group confirmed by MS. The solution was filtered with celite, and evaporated to provide O-methyl-L-Lys-carbonyldihydrofumagillol as a colorless oil (0.15 g).

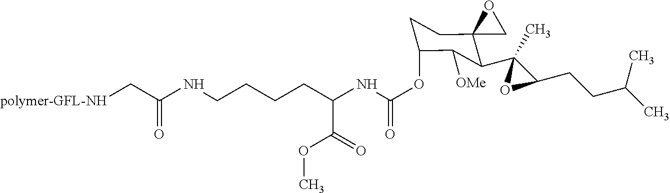

To a stirred solution of O-methyl-L-Lys-carbonyldihydrofumagillol (150 mg, 0.32 mmol) in DMF (6 mL) was added poly(HPMA-co-MA-GFLG-ONp) (300 mg) at 0° C. The resulting yellow solution was allowed to warm to room temperature overnight. The solvent was evaporated and the residue suspended in water (30 mL). The suspension was extracted six times with ethyl acetate (total ethyl acetate volume=150 mL). The aqueous phase was lyophilized to provide the polymer conjugate as a white solid (180 mg, 63%).

### Example 37

### Aminothiophenol Conjugate of Polymer and MetAP2Inhibitor Moiety

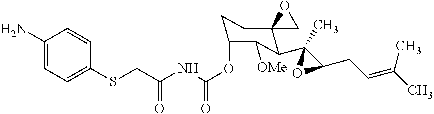

To a solution of chloroacetylcarbamoylfumagillol (500 mg) and 4-aminothiophenol (180 mg) in DMF (10 mL) at 0° C. was added DIEA (193 mg). The solution was stirred at 0° C. for 1.5 hours and then at room temperature overnight. The solution was diluted with water and extracted with ethyl acetate. Purification by flash chromatography (MeOH/CH₂Cl₂) followed by a second chromatography (EtOAc/hexanes) gave 4-aminophenylthioacetylcarbamoylfumagillol (460 mg).

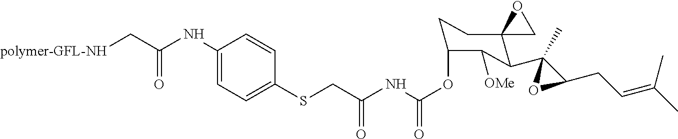

To a solution of poly(HPMA-co-MA-GFLG-ONp) (200 mg) and 4-aminophenylthioacetylcarbamoylfumagillol (100 mg) in DMF (5 mL) at 0° C. was added DIEA (106 mg). The solution was allowed to warm to room temperature and then heated to 50° C. and stirred overnight. The solvent was evaporated and the residue suspended in water. The suspension was extracted with ethyl acetate (150 mL). The aqueous phase was lyophilized to yield the polymer conjugate as a white solid (180 mg).

### Example 38

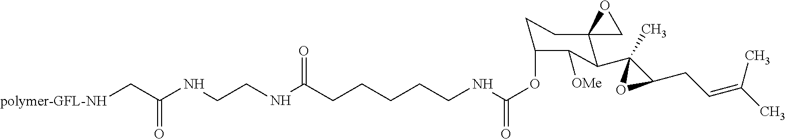

To a solution of poly(HPMA-co-MA-GFLG-NHCH₂CH₂NH₂.HCl) (200 mg) and N-(5-carboxypentyl)carbamoylfumagillol (96 mg) in DMF (6 mL) at 0° C. was added DIEA (104 mg) followed by N-(3-dimethylaminopropyl)-N′-ethylcarbodiimide hydrochloride (42 mg). The solution was allowed to warm to RT and stirred overnight. The solvent was evaporated and the residue dissolved in water (50 mL) and extracted with ethyl acetate (200 mL). The aqueous phase was purified by TFF with water (450 mL). The retentate was lyophilized to yield the polymer (200 mg) as a pale yellow solid.

### Example 39

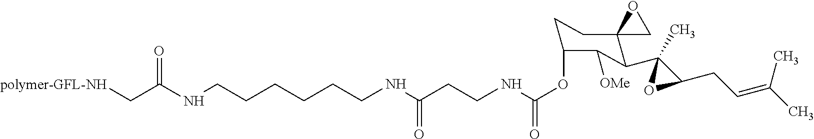

To a solution of poly[HPMA-co-MA-GFLG-N(CH₂)₆NH₂.HCl] (216 mg), 2-carboxyethylcarbamoylfumagillol (91 mg) in DMF (8 mL) at 0° C. was added DIEA (118 mg) followed by N-(3-Dimethylaminopropyl)-N′-ethylcarbodiimide hydrochloride (88 mg). The solution was allowed to warm to room temperature and stirred overnight. The solvent was evaporated and the residue dissolved in water (50 mL) and extracted with ethyl acetate (200 mL). The aqueous phase was purified by TFF (10 K) with water (1 L). The retentate was lyophilized to yield the polymer (170 mg) as a pale yellow solid.

### Example 40

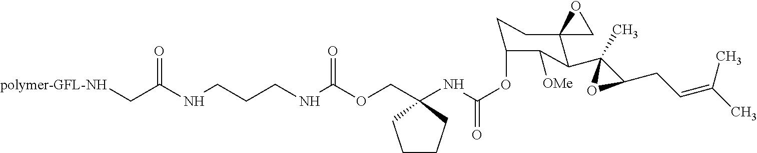

General Procedure F was followed using poly(HPMA-co-MA-GFLG-NHCH₂CH₂CH₂NH₂.HCl) (220 mg) and carbonate (Example 24, 100 mg) in DMF (6 mL) with DIEA (63 mg). The reaction was extracted with ethyl acetate. Following TFF (10 K) purification with water, and lyophilization, the product was isolated as a light pink powder (140 mg).

### Example 41

General Procedure F was followed using poly(HPMA-co-MA-GFLG-NHCH₂CH₂CH₂NH₂.HCl) (200 mg) and carbonate (Example 23, 86 mg) in DMF (5 mL) with DIEA (57 mg). Extraction was performed with ethyl acetate. Following TFF purification with water, and lyophilization, the product was isolated as a light pink powder (200 mg).

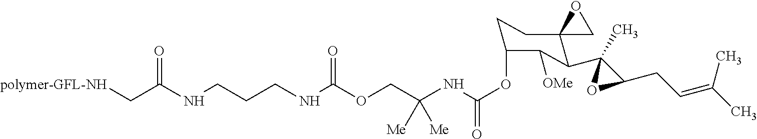

### Example 42

### Synthesis of poly[HPMA-co-MA-GFLG-N-(6-aminohexyl)acetamide]

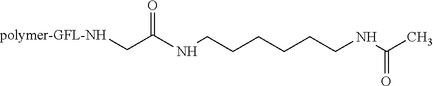

To a solution of aminohexylpolymer (600 mg) and p-nitrophenyl acetate (110 mg) in DMF (16 mL) at 0° C. was added DIEA dropwise. The solution was allowed to warm to room temperature and stirred overnight. The solvent was evaporated and the residue was dissolved in water (50 mL), filtered through a vacu-cap filter with an additional 25 mL of water. The pH was adjusted to 8.0 with 0.1 M NaOH and the solution concentrated to 50 mL (TFF). The retentate was washed with aqueous NaCl (25 mM, 450 mL) until the permeate was almost colorless and then washed with water (400 mL) to a conductivity of 0.00 uS. The retentate was lyophilized to yield 0.59 g of a pink solid.

### Example 43

### Aqueous Stability of Carbamoylfumagillol

A stock solution of carbamoylfumagillol in DMSO was diluted in a 15 mL polypropylene screw top tube with either 5 mL of 10 mM sodium acetate buffer at either pH 4.0 or 5.3, or potassium phosphate buffer at pH 6.7 or 8.0 at 37° C. The final concentration of carbamoylfumagillol in the buffer solution was 5 μM. At the appropriate time points, a 50 μL sample was withdrawn and diluted with three volumes of methanol containing propranolol as an internal standard (one solution was made for the entire study). The concentration of carbamoylfumagillol in the solution was analyzed by LC/MS/MS over seven days. From pH 5.3 to 8.0, less than 20% decomposition was observed over the seven day period. Estimated rate constants are presented in Table 1.

TABLE 1
Natural Rate Constant of Carbamoylfumagillol after Incubation 37° C.
in Aqueous Buffer at Various pHs

| pH | Natural Rate Constant (hr⁻¹) | T ½ (hr) |
|---|---|---|
| 4.0 | 0.0054 | 129 |
| 5.3 | 0.0017 | 407 |
| 6.7 | 0.0010 | 728 |
| 8.0 | 0.0011 | 613 |

*the values in italics are approximate as the decompositions did not reach 50% in 168 hours
**The half life is calculated as ln(2)/rate constant.

### Example 44

### Water in Polymer Conjugates

Selected polymers were analyzed by Karl Fisher (QTI Salem Industrial Park—Bldg. #5 Whitehouse, N.J. 08888) to determine the water content of the polymer. The results are summarized below in Table 2.

TABLE 2

| Sample | Water Content % |
|---|---|
| O-7175 | 6.56 |
| O-7320 | 9.65 |
| O-7271 | 6.71 |
| O-7376 | 5.13 |

### Example 45

### Reaction of Carbamoylfumagillol with 2-Mercaptopyrimidine

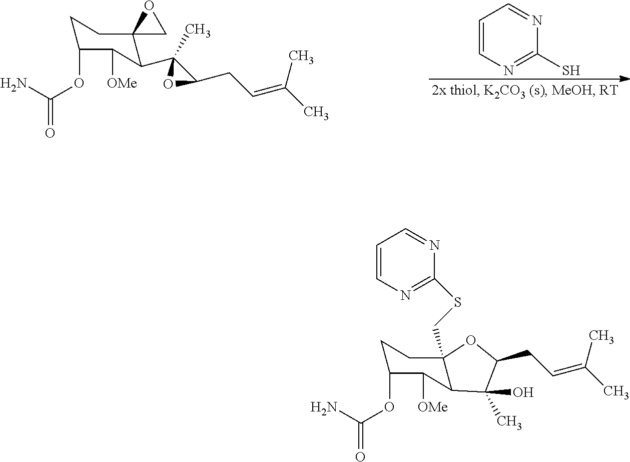

A stock solution, 1 mg/mL, of 2-mercaptopyrimidine (2.2 mL) in methanol-D4 was added to carbamoylfumagillol (6.4 mg). One mL of the resulting solution was removed and a second portion of the stock solution was added (1 mL). Solid K₂CO₃ was added and the solution monitored by ¹H NMR. A single product was identified, the 1:1 adduct of 2-mercaptopyrimidine and carbamoylfumagillol.

The following resonances were used to monitor the reaction by ¹H NMR:

2-Mercaptopyrimidine showed resonances at 6.7 ppm (1H, H-4) and 8.1 ppm (2H, H-3, H-5).

The adduct of 2-mercaptopyrimidine showed resonances at 7.2 ppm (1H, H-4) and 8.5-8.6 ppm (2H, H-3, H-5).

### Example 46

### Reaction of Polymer Conjugates with 2-Mercaptopyrimidine

A stock solution, 1 mg/mL, of 2-mercaptopyrimidine (1.1 mL) in methanol-D4 was added to the polymer conjugate (10 mg). The solution was stirred at room temperature overnight, and analyzed by ¹H NMR to determine the ratio of unreacted thiol (8.1 ppm) to reacted thiol (8.5-8.6 ppm). The amount of reacted thiol was expected to be equivalent to the quantity of fumagillol in the polymer conjugate. The acetamide capped polymer containing no epoxide showed no reaction product with 2-mercaptopyrimidine as indicated in Table 3.

TABLE 3

| Sample | Reacted thiol/g polymer | |
|---|---|---|
| O-7175 | 0.37 | mmols/g |
| O-7320 | 0.37 | mmols/g |
| O-7376 | <0.001 | mmols/g |

### Example 47

### Cathepsin B Release of Fumagillol Analogs

Cathepsin B (Sigma Cat #C6286 Lot #025K7672) was diluted to a 10× concentration in activation buffer consisting of approximately 400 nM enzyme, 30 mM DTT, 15 mM EDTA and acetate buffer, pH=5.5 for 15 minutes at room temperature.

The HPMA conjugates were made into a 10x stock solution in pH 5.5 buffer. The final reaction was performed by diluting the enzyme and substrate 10 fold into either buffer at pH=5.5 or pH=6.8. The final enzymatic reaction consisted of 40 nM Cathepsin B, approximately 2.5 mg/mL test agent, and buffer at 37° C. The reaction was stopped at 0, 2, 6, and 24 hour. To stop the reaction, 3 volumes of ice-cold methanol containing propranolol internal standard (at 1.0 μM) was added and left on ice. The samples were then analyzed by LC/MS/MS.
poly[HPMA-co-MA-GFLG-N-(6-aminohexyl)carbamoylfumagillol] was shown to release N-(6-aminohexyl)carbamoylfumagillol and fumagil-6-yl {6-[(aminoacetyl)amino]hexyl}carbamate.
poly(HPMA-co-MA-GFLG-NHCH₂CH₂N(Me)CH₂C(O)NHC(O)₂-fumagill-6-yl) was shown to release fumagillol, carbamoylfumagillol, and fumagil-6-yl (2-aminoethyl)methylcarbamate.
poly(HPMA-co-MA-GFLG-N(Me)CH₂CH₂N(Me)CH₂C(O)NHC(O)₂-fumagill-6-yl) was shown to release fumagillol, carbamoylfumagillol, fumagil-6-yl methyl[2-(methylamino)ethyl]carbamate, and ethyl{2-[(aminoacetyl)(methyl)amino]ethyl}methylcarbamate.

### Example 48

### General Materials and Methods for In Vitro Analysis

Test compounds, small molecules or polymer conjugates, were dissolved in dimethyl sulfoxide to a stock concentration of 5 mg/mL. The test agents were then diluted to an intermediate concentration at 200 μg/mL in 10% DMSO. Further dilutions were completed serially 3-fold in 10% DMSO to produce 12 decreasing concentrations for in-vitro analysis. To achieve the target concentrations of the in-vitro assays, 1 μL of the intermediate drug preparation was delivered to the cells (seeded in a volume of 50 μL). The final DMSO concentration for the tests was 0.2% for all doses of test agent.

Cells were exposed to twelve increasing concentrations of formulated test agent from 2×10⁻⁶ to 4.0 μg/mL for 72 hours. Following 72 hour exposure, 25 μL it of CellTiter-Glo® Reagent was added to each well. The plates were incubated for 60 minutes at 37° C. in a humidified incubator. After incubation, luminescence was recorded using the Molecular Devices AnalystGT multi-mode reader.

### IC50 Determination

Data are expressed as the percent cell growth of the untreated (vehicle) control calculated from the luminescence signals. The surviving fraction of cells is determined by dividing the mean luminescence values of the test agents by the mean luminescence values of untreated control. The inhibitory concentration value for the test agent(s) and control were estimated using Prism 5 software (GraphPad Software, Inc.) by curve-fitting the data using the non-linear regression analysis.

### Example 49

### A549 Human Non-Small Cell Lung Carcinoma Cell Viability Assay

The human tumor cell lines A549 and HCT-116 were obtained from American Type Culture Collection (Manassas, Va.). The Human umbilical vein epithelial cells (HUVEC) were obtained from Lonza (Basel, Switzerland). The A549 cells were maintained RPMI 1640 w/L-glut supplemented with 5% FBS. The HCT-116 cells were maintained in McCoy's 5a supplemented with 5% FBS. The HUVEC line was grown in Endothelial Growth Medium with supplements and growth factors (BBE, hydrocortisone, hEGF, FBS and gentamicin/amphotericin-B). All cells were house in an atmosphere of 5% CO₂ at 37° C. Cells were dissociated with 0.05% Trypsin and 0.02% EDTA.

The human tumor cell line A549 was obtained from American Type Culture Collection (Manassas, Va.). The A549 cells were maintained RPMI 1640 w/L-glut supplemented with 5% FBS. A549 cells were seeded at 500 cells per well 24 hours prior to test agent exposure in a volume of 50 μL. The cells were housed in an atmosphere of 5% CO₂ at 37° C. Cells were dissociated with 0.05% Trypsin and 0.02% EDTA.

TABLE 4
A549-Small Molecules

| A549 IC50 | Compound # |
|---|---|
| Average ng/mL | |
| 0.508 | O-7233 |
| 0.777 | O-7299 |
| 1.50 | O-7322 |
| 5.99 | O-7319 |
| 23.2 | O-7287 |
| 0.215 | O-7177 (PPI-2458) |
| 1.06 | O-7216 |
| 2.89 | O-7127-1 |
| | (Carbamoylfumagillol) |
| 8.97 | O-7178 (TNP-470) |
| 30.1 | O-7126-1 (Fumagillol) |

TABLE 5
A549-Polymer Conjugates

| A549 IC50 | Compound # |
|---|---|
| Average μg/mL | |
| 0.86 | O-7172 |
| 1.08 | O-7173 |
| 0.40 | O-7174 |
| 0.50 | O-7175 |
| 2.57 | O-7176 |
| 1.11 | O-7192 |
| 0.28 | O-7193 |
| 1.12 | O-7195 |
| 0.67 | O-7196 |
| 0.12 | O-7215 |
| 0.52 | O-7232 |
| 0.40 | O-7234 |
| 1.16 | O-7271 |
| 0.08 | O-7272 |
| 0.17 | O-7303 |
| 0.42 | O-7304 |
| 4.00 | O-7305 |
| 0.89 | O-7306 |
| 0.32 | O-7320 |
| 0.42 | O-7321 |
| 0.98 | O-7323 |
| 1.54 | DRS-226-46E |

### Example 50

### HCT-116 Human Colon Tumor Cell Viability Assay

The human tumor cell lines A549 and HCT-116 were obtained from American Type Culture Collection (Manassas, Va.). The HCT-116 cells were maintained in McCoy's 5a supplemented with 5% FBS. HCT-116 cells were seeded at 500 cells per well 24 hours prior to test agent exposure in a volume of 50 μL. The cells were housed in an atmosphere of 5% CO₂ at 37° C. Cells were dissociated with 0.05% Trypsin and 0.02% EDTA.

Cells were exposed to twelve increasing concentrations of formulated test agent from 2.3×10⁻⁶ to 4.02 μg/mL for 72 hours. Following 72 hour exposure, 25 μL of CellTiter-Glo® Reagent was added to each well. The plates were incubated for 60 minutes at 37° C. in a humidified incubator. After incubation, luminescence was recorded using the Molecular Devices AnalystGT multi-mode reader.

TABLE 6
HCT116-Small Molecules

| HCT116 IC50 | Compound # |
|---|---|
| Average ng/mL | |
| 0.236 | O-7177 |
| 0.408 | O-7194 |
| 0.918 | O-7216 |
| | O-7127-1 |
| 1.035 | (Carbamoylfumagillol) |
| 2.64 | O-7178 (TNP-470) |
| 45.8 | O-7126-1 (Fumagillol) |

TABLE 7
HCT116-Polymer Conjugates

| HCT116 IC50 | Compound # |
|---|---|
| Average ug/mL | |
| 0.157 | O-7215 |
| 0.329 | O-7193 |
| 0.392 | O-7174 |
| 0.626 | O-7175 |
| 0.818 | O-7196 |
| 1.221 | O-7172 |
| 1.051 | O-7173 |
| 1.184 | O-7192 |
| 1.203 | O-7195 |
| 0.984 | DRS-226-46E |
| 5.954 | O-7176 |

### Example 51

### Human Umbilical Vein Epithelial Cell Viability Assay

The Human umbilical vein epithelial cells (HUVEC) were obtained from Lonza (Basel, Switzerland). The HUVEC line was grown in Endothelial Growth Medium with supplements and growth factors (BBE, hydrocortisone, hEGF, FBS and gentamicin/amphotericin-B). All cells were housed in an atmosphere of 5% CO₂ at 37° C. Cells were dissociated with 0.05% Trypsin and 0.02% EDTA.

HUVEC cells were seeded at 1000 cells per well 24 hours prior to test agent exposure in a volume of 50 μL. Cells were exposed to twelve increasing concentrations of formulated test agent from 2.3×10⁻⁶ to 4.02 μg/mL for 72 hours. Following 72 hour exposure, 25 μL of CellTiter-Glo® Reagent was added to each well. The plates were incubated for 60 minutes at 37° C. in a humidified incubator. After incubation, luminescence was recorded using the Molecular Devices AnalystGT multi-mode reader.

TABLE 8
HUVEC-Small Molecules

| HUVEC IC50 | Compound # |
|---|---|
| Average ng/mL | |
| 0.101 | O-7177 |
| 0.120 | O-7194 |
| 0.209 | O-7216 |
| 0.086 | O-7127-1 |
| | (Carbamoylfumagillol) |
| 0.153 | O-7178 (TNP-470) |
| 18.9 | O-7126-1 (Fumagillol) |

TABLE 9
HUVEC-Polymer Conjugates

| HUVEC IC50 | Compound # |
|---|---|
| Average ug/mL | |
| 0.157 | O-7215 |
| 0.329 | O-7193 |
| 0.392 | O-7174 |
| 0.626 | O-7175 |
| 0.818 | O-7196 |
| 1.221 | O-7172 |
| 1.051 | O-7173 |
| 1.184 | O-7192 |
| 1.203 | O-7195 |
| 0.984 | DRS-226-46E |
| 5.954 | O-7176 |

### Example 52

### A549/HUVEC Selectivity

The ratio of the HUVEC IC50/A549 IC₅₀ is presented in Table 10 below. When compared to carbamoylfumagillol and TNP-470, the polymer conjugates are more active against the tumor cells, A549, than against the normal HUVEC cells.

TABLE 10

| A549/HUVEC IC50 | Compound # |
|---|---|
| IC50 ratio | |
| 2.14 | O-7177 |
| 2.97 | O-7194 |
| 5.06 | O-7216 |
| | O-7127-1 |
| 33.63 | (Carbamoylfumagillol) |
| 58.53 | O-7178 (TNP-470) |
| 1.59 | O-7126-1 (fumagillol) |
| Polymer Conjugates | |
| 0.66 | O-7215 |
| 3.13 | O-7193 |
| 2.04 | O-7174 |
| 2.64 | O-7175 |
| 2.26 | O-7196 |
| 1.52 | O-7172 |
| 1.30 | O-7173 |
| 1.81 | O-7192 |
| 1.66 | O-7195 |
| 4.33 | DRS-226-46E |
| 0.98 | O-7176 |

### Example 53

### A549 Metabolites

Cells were treated as in Example 51 except that at the end of 72 hour exposure to test agent, the cells were frozen (−70° C.) and stored for subsequent evaluation by LC/MS. Metabolites identified from the cells treated with poly[HPMA-co-MA-GFLG-N-(6-aminohexyl)carbamoylfumagillol] include N-(6-aminohexyl)carbamoylfumagillol, fumagill-6-yl {6-[(aminoacetyl)amino]hexyl}carbamate, and the epoxide hydrolysis product, (3S,7aR)-7a-(hydroxymethyl)-4-methoxy-3-methyl-2-(3-methylbut-2-en-1-yl)octahydro-1-benzofuran-3-ol-5-yl 6-aminohexyl carbamate.

### Example 54

### In Vivo Testing B16-F10 Murine Melanoma

C57B16 female mice (N=8) were injected (tail vein) with 1×10⁵ B16-F10 tumor cells. After one day, mice were treated with polymer conjugates as solutions in saline (IV administration, q4d, four doses except that in one group O-7175 was administered as a single dose on day 1). TNP-470 was used as a positive control, saline as a negative control. Mice were sacrificed after 15 days. Treatment outcomes were assessed by counting lung metastases.

TABLE 11
Metastases Counts

| | | Metastases |
|---|---|---|
| Group | Dose mg/kg* | Counts |
| Saline control | 0 | 36.8 |
| TNP-470 | 30 | 39.5 |
| O-7175 | 50 | 17.0 |
| O-7175 | 100 | 24.5 |
| O-7175 | 200 | 20.9 |
| O-7320 | 200 | 7.6 |
| O-7271 | 200 | 20.0 |
| O-7215 | 200 | 32.5 |
| O-7175 | 1000 | 10.1 |

*All groups, N = 8. IV dosing q4d, days 1, 5, 9 and 13 except TNP-470 (qod) and O-7175 at 1000 mg/kg (single dose on day 1).

### Example 55

### In vivo Testing C57Bl6 Mice—Weight Changes

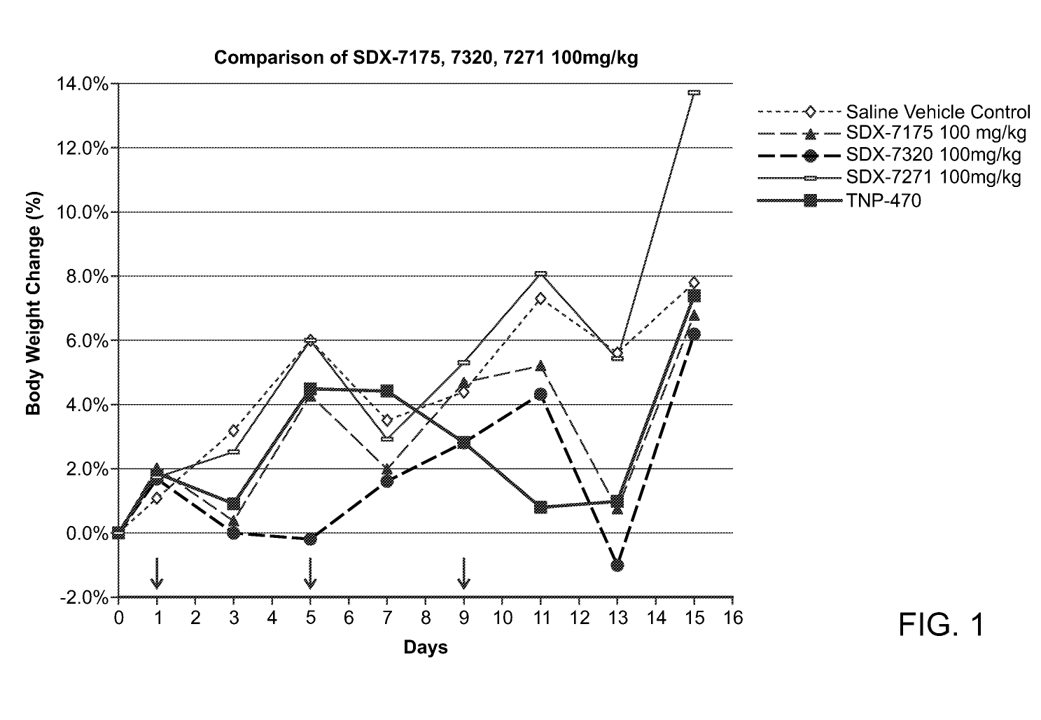

C57B16 female mice (N=8) were injected (tail vein) with 1×10⁵ B16-F10 tumor cells. After one day, mice were treated with polymer conjugates as solutions in saline (IV administration, q4d, four doses). The weight changes for three polymers relative to saline vehicle control and TNP-470 are shown in FIG. 1. Weight changes are referenced to the group weight at time zero. All polymers were dosed at 100 mg/kg. Polymer doses and the saline vehicle were administered on days 1, 5, and 9. The 100 mg/kg polymer doses and TNP-470 showed a reduction in metastases from 44-63% relative to the saline control.

### Example 56

### In Vivo Testing C57Bl6 Mice—Weight Changes

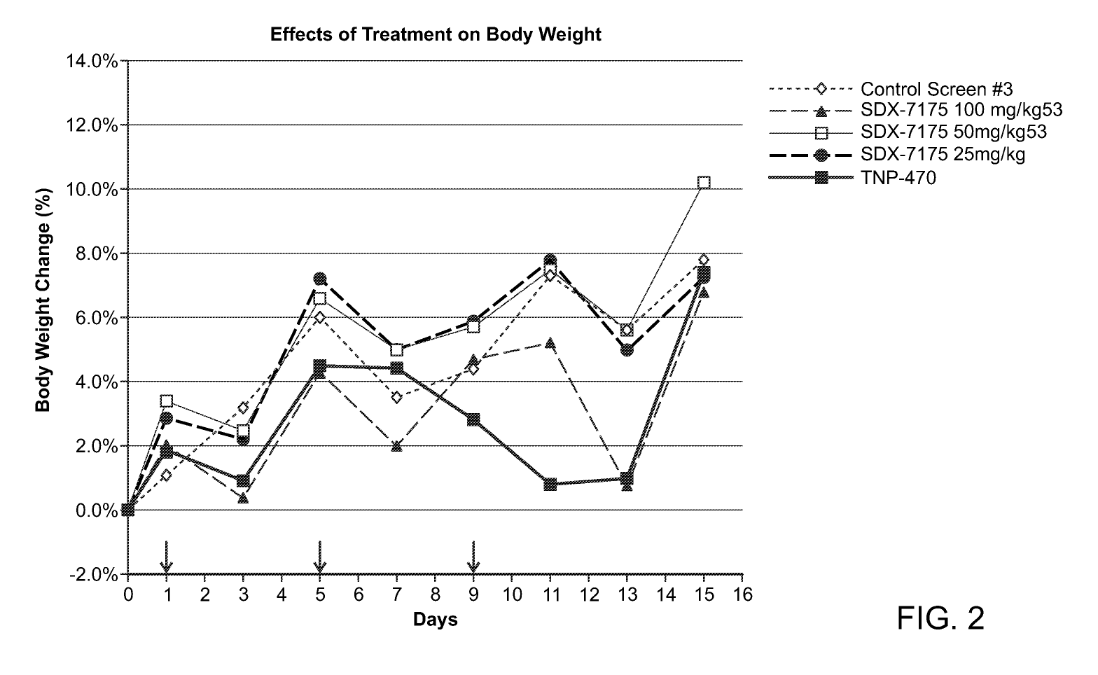

C57B16 female mice (N=8) were injected (tail vein) with 1×10⁵ B16-F10 tumor cells. After one day, mice were treated with polymer conjugates as solutions in saline (IV administration, q4d, four doses). The weight changes for one polymer at three different doses relative to control are shown in FIG. 2. Weight changes are referenced to the group weight at time zero. The polymer doses were 50 mg/kg, or 100 mg/kg. Polymer doses were administered on days 1, 5, and 9. The 25, 50 and 100 mg/kg polymer doses and TNP-470 showed a reduction in metastases from 45-61% relative to the saline control.

### Example 57

### In Vivo Testing nu/nu Mice—A549 Xenograft

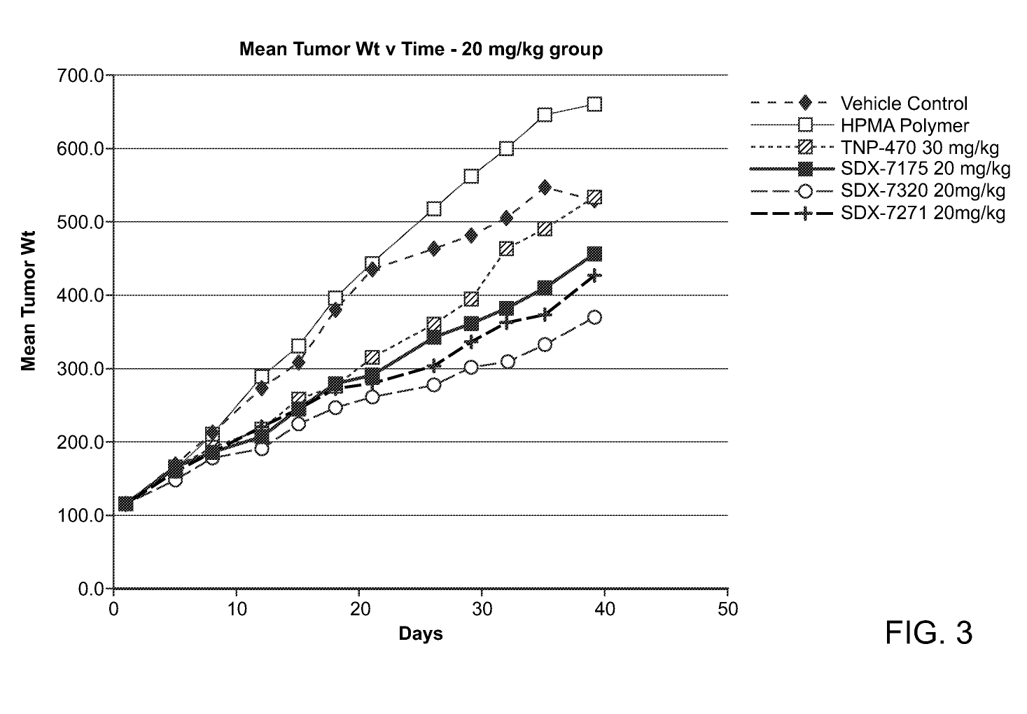

Nu/nu female mice (N=8) were injected (subcutaneous right flank) with 5×10⁶ A549 tumor cells (inoculation vehicle 50% media/matrigel, subcutaneous right flank). After the tumors reached a size of 116 mg, mice were treated with polymer conjugates as solutions in saline (20 mg/kg, IV administration, q4d, six doses) or with a control polymer without a MetAP2 inhibitory moiety (100 mg/kg, q4d) or with TNP-470 (30 mg/kg, qod, nine doses). Tumor growth was determined by measuring tumor size in two directions with calipers at intervals of a few days. The tumor size vs time is shown in FIG. 3. The doses used are summarized in the table below.

TABLE 12

| | | | Single Dose | Total Dose | Total Dose |
|---|---|---|---|---|---|
| | Schedule | # doses | mg/kg | mg | mmol active |
| TNP-470 | qod | 9 | 30 | 270 | 0.67 |
| Polymer | q4d | 6 | 20 | 120 | 0.044 |
| | frequency | # doses | wt/wt | wt/wt | mol/mol |
| polymer % | 50% | 67% | 67% | 44% | 7% |

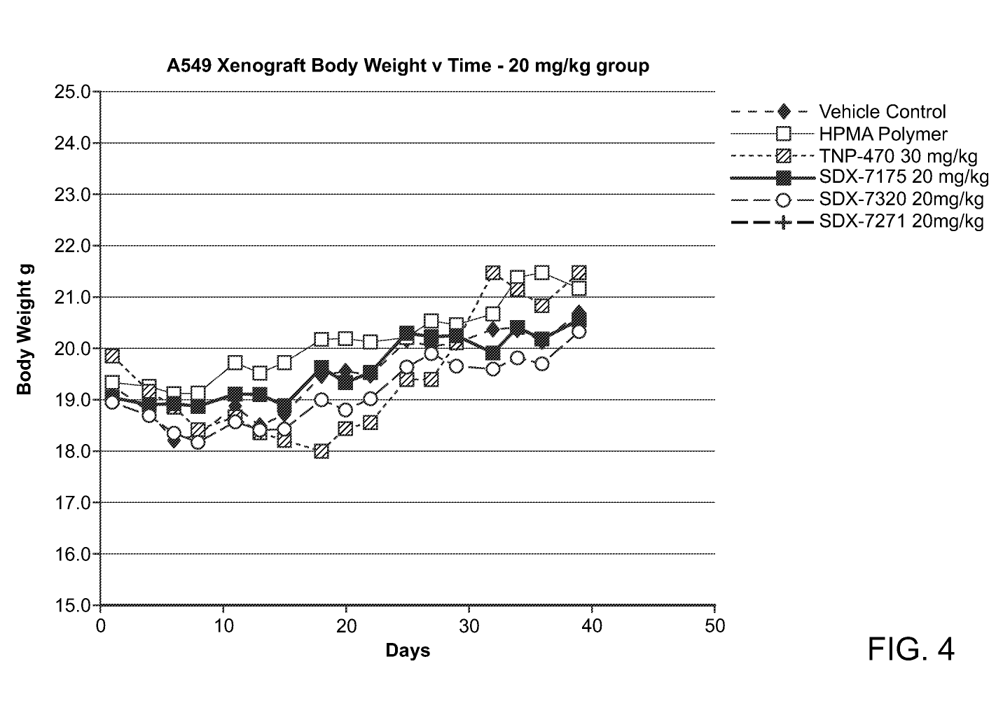

The change in body weight vs time for the A549 Xenograft experiment is shown in FIG. 4. The mice in the active polymer treated groups show similar weight changes to the TNP-470 group and the control groups.

### Example 58

### In Vivo Testing nu/nu Mice—A549 Xenograft

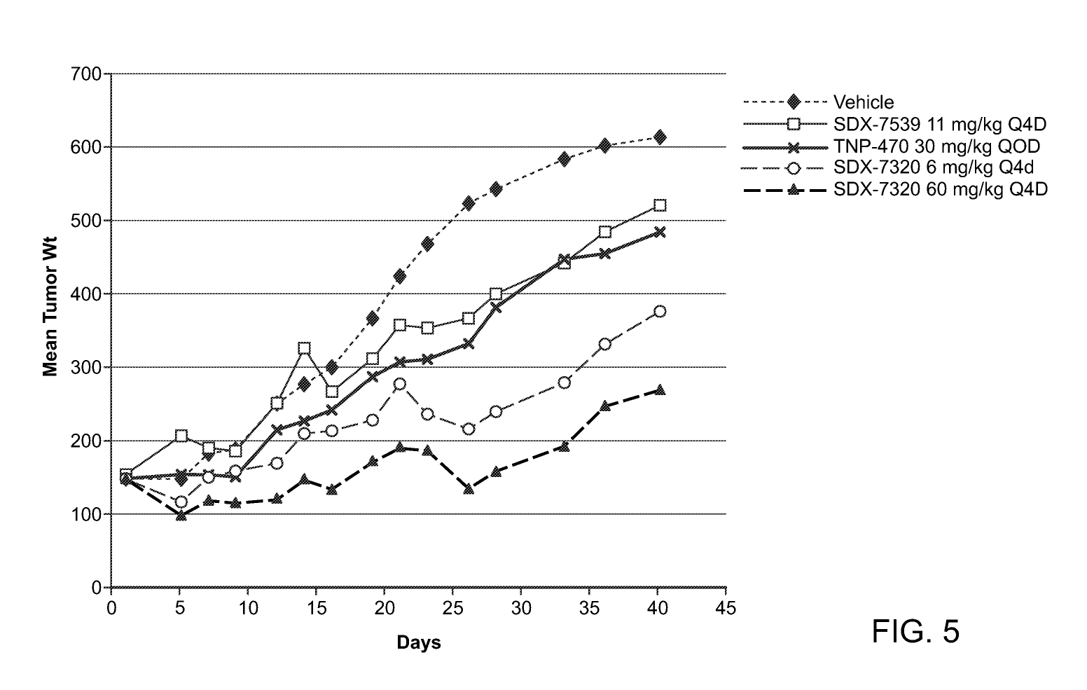

Nu/nu female mice (N=8) were injected (subcutaneous right flank) with 5×10⁶ A549 NSCLC cells (inoculation vehicle 50% media/matrigel, subcutaneous right flank). After the tumors reached a size of 150 mg, mice were treated with a polymer conjugate as a solutions in saline at a dose level of either 6 mg/kg or 60 mg/kg (IV administration, q4d, seven doses) or with a small molecule, the active metabolite released from the polymer conjugate, (11 mg/kg, IV administration, q4d, seven doses) or with TNP-470 (30 mg/kg, qod, nine doses). Tumor growth was determined by measuring tumor size in two directions with calipers at intervals of a few days. The tumor size versus time is shown in FIG. 5. A comparison of the polymer conjugate dose to the TNP-470 dose is shown in Table 13 below.

TABLE 13

| | | | Single | Total | |
|---|---|---|---|---|---|
| | | | Dose | Dose | Total Dose |
| | Schedule | # doses | mg/kg | mg | mmol active |
| TNP-470 | qod | 9 | 30 | 270 | 0.67 |
| Polymer (low dose) | q4d | 7 | 6 | 42 | 0.044 |
| | frequency | # doses | wt/wt | wt/wt | mol/mol |
| polymer % | 50% | 78% | 20% | 16% | 7% |

The low polymer dose is more active than TNP-470 at a total dose less than 3 mole % of the TNP-470 dose.

### Example 59

### In Vivo Testing Pharmacokinetics of Polymer Conjugate

The polymer conjugate, SDX-7320, or the in vivo release product, SDX-7539 were administered to Sprague-Dawley rats (N=3). Blood was collected over 48 hours after dosing to determine the plasma concentration of SDX-7539 by LC-MS/MS. The LLOQ for SDX-7539 was 2.5 nM. The terminal elimination half-life for SDX-7539 was estimated by fitting a linear regression to the ln [SDX-7539] versus time data. The half-life of the small molecule SDX-7539 is in the range of 10-15 minutes; Cₘₐₓ is approximately 15 μM and occurs at T₀. For the polymer conjugate, SDX-7320, the released small molecule exhibits a Cₘₐₓ of approximately 0.3 μM at 2 hours and a terminal elimination half-life of 10 hours. Cₘₐₓ for the polymer is about 2% of the value for the small molecule. The AUC for SDX-7539 resulting from either administration of SDX-7539, itself, or SDX-7320 are comparable.

What is claimed is:

1. A drug conjugate composition, comprising:
an active moiety that is modified to have reduced efflux from a target tissue compared to an unmodified active moiety;
a conjugate moiety; and
a cleavable linker;
wherein cleavage of the linker occurs in target tissue to release said modified active moiety.

2. The composition according to claim 1, wherein the modification comprises a change in molecular weight of the active moiety.

3. The composition according to claim 1, wherein the modification comprises a change in charge of the active moiety.

4. The composition according to claim 1, wherein the modification comprises a change in hydrophobicity of the active moiety.

5. The composition according to claim 1, wherein the modification comprises a change in polar surface area of the active moiety.

6. The composition according to claim 1, wherein the active moiety is modified prior to linkage to the conjugate moiety.

7. The composition according to claim 1, wherein the linker comprises a structure that provides for intracellular proteolytic cleavage.

8. The composition according to claim 1, wherein the conjugate moiety is selected from a polymer, a peptide, an antibody, a nucleic acid, and an aptamer.

9. The composition according to claim 1, wherein the conjugate moiety is biocompatible.

10. The composition according to claim 1, wherein the conjugate moiety does not accumulate.

11. The composition according to claim 1, wherein the conjugate moiety is non-immunogenic.

12. The composition according to claim 1, wherein the conjugate moiety is hydrophilic.

13. The composition according to claim 1, wherein the conjugate moiety is biodegradable.

14. The composition according to claim 13, wherein the conjugate moiety degrades at a rate that is slower than the rate of release of the modified active moiety.

15. The composition according to claim 1, wherein the conjugate moiety is not biodegradable.

16. The composition according to claim 1, wherein the active moiety is a molecule that produces a therapeutic effect in a subject.

17. The composition according to claim 1, wherein the active moiety is an anticancer drug.

18. The composition according to claim 1, wherein the active moiety is a molecule that inhibits MetAP2 activity.

19. The composition according to claim 1, wherein the active moiety is an analog or derivative of fumagillol.

20. A drug conjugate composition, the composition comprising:
an active moiety;
a conjugate moiety; and
a cleavable linker;
wherein cleavage of the linker occurs substantially in target tissue to release a modified active moiety having reduced efflux from target tissue compared to an unmodified active moiety.

21. The composition according to claim 20, wherein the modified active moiety comprises the active moiety having a fragment of the cleaved linker attached thereto.

22. A drug conjugate composition, the composition comprising:
an active moiety that has low or no capability to enter a cell;
a conjugate moiety; and
a cleavable linker,
wherein cleavage of the linker occurs substantially in a target tissue and the cleaved active moiety is released intracellularly.

23. A drug conjugate composition, the composition comprising:
an active moiety that is modified to have reduced efflux from a target tissue compared to an unmodified active moiety;
a conjugate moiety; and
a cleavable linker,
wherein cleavage of the linker occurs in a target tissue and the modified active moiety is inactivated in the target tissue at a higher rate than it is transported out of the target tissue.

24. A drug conjugate composition, the composition comprising:
an active moiety that is modified to have reduced efflux from a target tissue compared to an unmodified active moiety;
a conjugate moiety; and
a cleavable linker,
wherein cleavage of the linker occurs in the target tissue and the modified active moiety is metabolized more rapidly in the tissue than its transport rate away from tissue.

25. A drug conjugate composition, the composition comprising:
an active moiety that is modified to have reduced efflux from a target tissue compared to an unmodified active moiety;
a conjugate moiety; and
a cleavable linker,
wherein cleavage of the linker occurs in the target tissue and the modified active moiety has at least five-fold greater pharmaceutical activity in the tissue as compared to the active moiety alone.

26. An optimized drug composition, the composition comprising
an active drug moiety; and
a portion of a cleavable linker,
wherein a majority of the composition is retained and inactivated in a tissue to which it is targeted, and wherein amounts of the composition that diffuse away from the tissue are metabolized at a greater rate than the active moiety alone.
